(19) United States
(12) Patent Application Publication
(10) Pub. No.: US 2023/0002731 A1
(43) Pub. Date: Jan. 5, 2023

(54) METHOD OF PRODUCING NATURAL KILLER CELLS AND COMPOSITIONS THEREOF

(71) Applicant: NKMAX Co., Ltd., Gyeonggi-do (KR)

(72) Inventors: Sang Woo Park, Gyeonggi-do (KR); Yong Man Kim, Gyeonggi-do (KR); Jae Seob Jung, Gyeonggi-do (KR); Yoonmi Kang, Gyeonggi-do (KR)

(21) Appl. No.: 17/780,204

(22) PCT Filed: Nov. 24, 2020
(86) PCT No.: PCT/US2020/061984

§ 371 Date: May 26, 2022

Related U.S. Application Data
(60) Provisional application No. 63/062,694, filed on Aug. 7, 2020.

(30) Foreign Application Priority Data
Nov. 29, 2019 (KR) 10-2019-0157727

(51) Int. Cl. C12N 5/0783

(52) U.S. Cl. CPC C12N 5/0646; C12N 2501/2321; C12N 2501/2302; C12N 2502/11

(57) ABSTRACT

A method for producing natural killer cells is disclosed. The method comprises isolating peripheral blood mononuclear cells (PBMCs) from a blood sample; isolating at least one of CD56+ cells and/or CD3−/CD56+ cells from the PBMCs; and co-culturing the at least one of CD56+ cells and/or CD3−/CD56+ cells with a combination of feeder cells in the presence of a cytokine. The method can further comprise freezing and thawing the CD56+ cells and/or CD3−/CD56+ cells. A composition for treating cancer is also disclosed. The composition comprises the CD56+ natural killer cells produced by the disclosed method and a cytokine.

### INCORPORATION BY REFERENCE TO ANY PRIORITY APPLICATIONS

This application claims the benefit of U.S. Provisional Application No. 63/062694, filed Aug. 7, 2020 and Korean Patent Application No. KR-10-2019-0157727, filed Nov. 29, 2019, the disclosure of each of which is hereby incorporated by reference in its entirety.

### BACKGROUND

### Field

The present disclosure relates to a manufacturing of, storing of and/or natural killer cells themselves.

### Description of the Related Art

Natural killer cells (NK cells) are one type of innate immune cells, which are known to non-specifically kill cancer, recognize and kill viruses, bacteria, and the like, and kill pathogens with enzymes such as perforin and granzyme or by Fas-FasL interaction. In the case of cancer patients, it has been reported that a decrease in cancer cell cytotoxicity of these NK cells is associated with the onset of various types of cancer, such as lung cancer (Carrega P, et al., Cancer, 2008: 112: 863-875), liver cancer (Jinushi M, et al., J Hepatol., 2005: 43; 1013-1020), breast cancer (Bauernhofer T, et al., Eur J Immunol., 2003: 33: 119-124), uterine cancer (Mocchegiani E., et al., Br j Cancer., 1999: 79: 244-250), blood cancer (Tajima F., et al, Lekemia 1996: 10: 478-482), and the like.

### SUMMARY

This application is related to methods of producing high-purity natural killer cells, and a cell therapeutic composition for treating cancer comprising high-purity natural killer cells and cytokines. Any features, structures, or steps disclosed herein can be replaced with or combined with any other features, structures, or steps disclosed herein, or omitted. Further, for purposes of summarizing the disclosure, certain aspects, advantages, and features of the inventions have been described herein. It is to be understood that not necessarily any or all such advantages are achieved in accordance with any particular embodiment of the inventions disclosed herein. No individual aspects of this disclosure are essential or indispensable.

In some embodiments, a method of expanding natural killer cells in culture is disclosed. The method includes isolating CD56+ cells from a blood sample; co-culturing the isolated CD56+ cells in the presence of IL-21 (and feeder cells) for a first period; freezing the co-cultured CD56+ cell after the first period; thawing the frozen CD56+ cells; and co-culturing the thawed CD56+ cells in the presence of IL-21 (and feeder cells) for a second period.

The method of any of the embodiments provided herein and/or any of the method disclosed herein can include one or more of the following features. The method can further include storing the frozen CD56+ cells at a temperature lower than −100° C. The method can further include storing the frozen CD56+ cells for more than a day before thawing. The isolated CD56+ cells can be co-cultured for between 13-16 days (or 9-25 days) before freezing. The isolated CD56+ cells can be co-cultured with one or more irradiated feeder cells in the presence of IL-21. The thawed CD56+ cells can be co-cultured with one or more irradiated feeder cells in the presence of IL-21. One or more feeder cells can be one or more selected from a group consisting of irradiated Jurkat cells, irradiated Epstein-Barr virus transformed lymphocyte continuous line (EBV-LCL) cells, K562 cells and PBMCs (including, for example, autologous PBMCs). The CD56+ cells can be co-cultured with a ratio of about 1:1-100 of CD56+ cells to feeder cells. In some embodiments, any feeder cell can be used for the first, second, or first and second expansion (e.g., with the use of 11-21). The CD56+ cells can be co-cultured with a ratio of about 1:1, 1:2, 1:5, 1:10, 1:20, 1:30 or 1:100 of CD56+ cells to feeder cells. In some embodiments, these ratios are for KL1/EBVLCL and for other feeder cells, 1:1 to 1:10 can be used, for example. IL-21 can be added at a concentration of 10-100 ng/mL during the first and/or second period. IL-21 can be added at a concentration of 20-80 ng/mL during the first and/or second period. IL-21 can be added at a concentration of 30-70 ng/mL during the first and/or second period. IL-21 can be added more than once during the first and/or second period.

In some embodiments, a method of expanding natural killer cells in culture is provided. The method includes isolating CD56+ from a blood sample (e.g., from PBMC, fresh or frozen, cord blood, and/or blood in which one has isolated CD56+, CD56+CD3−, and CD3− cells from a blood sample); co-culturing the CD56+ cells with one or more feeder cells in the presence of IL-21; freezing the CD56+ cells; thawing the frozen CD56+ cells; and expanding the thawed CD56+ cells (again, with any type of appropriate feeder cell).

The method of any of the embodiments and/or any of the method disclosed herein can include one or more of the following features. Freezing the CD56+ cells can be done at a temperature lower than −100° C. The method can further include storing the frozen CD56+ cells for a period more than a day and less than 10 years. The CD56+ cells can be co-cultured for between 13-16 (or 9-25) days before freezing. The one or more feeder cells is not limited in all embodiments and can be one or more selected from a group consisting of at least one of: irradiated Jurkat cells, irradiated Epstein-Barr virus transformed lymphocyte continuous line (EBV-LCL) cells, K562 cells, mb15-k562, mb21-k562 feeder cells, HuT78, and/or PBMCs. The CD56+ cells can be co-cultured with a ratio of about 1:1-100 of CD56+ cells to feeder cells. IL-21 can be added at a concentration of 10-100 ng/mL. IL-21 can be added more than once. In some embodiments, the NK cells can be used with any feeder cell type, as long as IL-21 is used prior to freezing, and as long as it is then followed by a restimulation process after thawing from the freezing.

In some embodiments, a method of increasing cytotoxicity of natural killer cells is disclosed. The method comprises providing said natural killer cells; freezing said natural killer cells; thawing the frozen natural killer cells; and co-culturing the thawed natural killer cells with one or more feeder cells in the presence of IL-21. Optionally, prior to freezing the natural killer cells, the natural killer cells can be co-cultured (expanded) with a feeder cell and IL-21.

The method of any of the preceding paragraphs and/or any of the method disclosed herein can include one or more of the following features. The method can further include storing the frozen natural killer cells at a temperature lower than −100° C. The method can further include storing the frozen natural killer cells for more than a day before thawing. The one or more feeder cells are one or more selected from a group consisting of irradiated Jurkat cells, irradiated Epstein-Barr virus transformed lymphocyte continuous line irradiated Jurkat cells, irradiated Epstein-Barr virus transformed lymphocyte continuous line (EBV-LCL) cells, K562 cells, mb15-k562, mb21-k562 feeder cells, HuT78, and/or PBMCs. The thawed natural killer cells can be co-cultured with a ratio of about 1:1-100 of CD56+ cells to feeder cells. IL-21 can be added at a concentration of 10-100 ng/mL. IL-21 can be added more than once.

In some embodiments, a method of treating a subject is disclosed. The method includes collecting CD56+ cells from the subject; co-culturing the CD56+ cells with one or more feeder cells in the presence of IL-21; freezing the co-cultured CD56+ cells for at least a day; thawing the frozen CD56+ cells; expanding the thawed CD56+ cells; and administering the expanded CD56+ cells to the subject, wherein the cytotoxicity of the cells from the second expansion is at least X% of a cytotoxicity of the co-cultured CD56+ before freezing.

The method of any of the preceding paragraphs and/or any of the method disclosed herein can include one or more of the following features. The method can further include storing the frozen CD56+ cells at a temperature lower than −100° C. The method can further include storing the frozen CD56+ cells for more than a day before thawing. The isolated CD56+ cells can be co-cultured for between 13-16 (or 9-25) days before freezing. Expanding the thawed CD56+ cells can include co-culturing the thawed CD56+ with one or more irradiated feeder cells in the presence of IL-21. The one or more feeder cells can be one or more selected from a group consisting of irradiated Jurkat cells, irradiated Epstein-Barr virus transformed lymphocyte continuous line (EBV-LCL) cells, K562 cells, mb15-k562, mb21-k562 feeder cells, HuT78, and/or PBMCs. The CD56+ cells can be co-cultured with a ratio of about 1:1-100 of CD56+ cells to feeder cells. IL-21 can be added at a concentration of 10-100 ng/mL during the first and/or second period. IL-21 can be added more than once during the first and/or second period.

In some embodiments, a composition is provided. The composition includes an effective amount of CD56+ cells derived from peripheral blood mononuclear cells (PBMCs) from the patient. The CD56+ cells are prepared by isolating peripheral blood mononuclear cells (PBMCs) from a blood sample; isolating CD56+ cells from the PBMCs; co-culturing the CD56+ cells with one or more feeder cells in the presence of one or more cytokines; freezing the CD56+ cells; thawing the frozen CD56+ cells; and co-culturing the thawed CD56+ cells with one or more feeder cells in the presence of one or more cytokines.

In some embodiments, a cell composition is disclosed. The cell composition includes: an effective amount of CD56+ cells derived from peripheral blood mononuclear cells (PBMCs) from the patient; IL-2; and IL-21.

In some embodiments, a composition is disclosed. The composition includes a first population of CD56+ cells derived from peripheral blood mononuclear cells (PBMCs); ice; IL-2; and IL-21. When thawed, the CD56+ cell has a cytotoxicity of at least 80% of a second population of CD56+ cells, wherein the second population of CD56+ cells have not been frozen.

In some embodiments, a method of expanding natural killer cells in culture is provided. The method can comprise: isolating CD56+ cells from a blood sample; co-culturing the isolated CD56+ cells in the presence of IL-21 for a first period; freezing the co-cultured CD56+ cell after the first period; thawing the frozen CD56+ cells; and co-culturing the thawed CD56+ cells in the presence of IL-21 for a second period.

In some embodiments, a method of expanding natural killer cells in culture is provided. The method comprises: isolating CD56+ from a blood sample; co-culturing the CD56+ cells with one or more feeder cells in the presence of IL-21; freezing the CD56+ cells; thawing the frozen CD56+ cells; and expanding the thawed CD56+ cells.

In some embodiments, a method of increasing cytotoxicity of natural killer cells is provided and comprises providing said natural killer cells; freezing said natural killer cells; thawing the frozen natural killer cells; and co-culturing the thawed natural killer cells with one or more feeder cells in the presence of IL-21.

In some embodiments, a method of treating a subject is provided and comprises: collecting CD56+ cells from the subject; co-culturing the CD56+ cells with one or more feeder cells in the presence of IL-21; freezing the co-cultured CD56+ cells for at least a day; thawing the frozen CD56+ cells; expanding the thawed CD56+ cells; and administering the expanded CD56+ cells to the subject, wherein the cytotoxicity of the cells from the second expansion is at least 80% of a cytotoxicity of the co-cultured CD56+ before freezing.

In some embodiments, a composition is provided and comprises: an effective amount of CD56+ cells derived from peripheral blood mononuclear cells (PBMCs) from the patient, wherein the CD56+ cells are prepared by: isolating peripheral blood mononuclear cells (PBMCs) from a blood sample; isolating CD56+ cells from the PBMCs; co-culturing the CD56+ cells with one or more feeder cells in the presence of one or more cytokines; freezing the CD56+ cells; thawing the frozen CD56+ cells; and co-culturing the thawed CD56+ cells with one or more feeder cells in the presence of one or more cytokines.

In some embodiments, a cell composition is provided and comprises: an effective amount of CD56+ cells derived from peripheral blood mononuclear cells (PBMCs) from the patient; IL-2; and IL-21.

In some embodiments, a composition is provided and comprises: a first population of CD56+ cells derived from peripheral blood mononuclear cells (PBMCs); ice; and IL-2, IL-21. When thawed, the CD56+ cell has a cytotoxicity of at least 80% of a second population of CD56+ cells and the second population of CD56+ cells have not been frozen.

In some embodiments, a method of expanding natural killer cells in culture is provided and comprises: providing PBMCs; co-culturing the PBMCs in the presence of IL-21 for a first period; freezing the co-cultured PBMCs after the first period; thawing the frozen PBMCs; and co-culturing the thawed PBMCs in the presence of IL-21 for a second period.

In some embodiments, the composition comprises: IL-2; 5-10% DMSO; 90-95% FBS; and NK cells that are optionally CD56+ cells. In some embodiments, it further comprises CryoStor solution. In some embodiments, the composition is for frozen cells before re-expansion.

In some embodiments, the composition comprises IL-2; 5-10% DMSO; 80-95% Hartman solution; 1-10% human serum albumin; and NK Cells. In some embodiments, it further comprises CryoStor solution. In some embodiments, the composition is for frozen cells before injection.

### BRIEF DESCRIPTION OF THE DRAWINGS

Various embodiments are depicted in the accompanying drawings for illustrative purposes, and should in no way be interpreted as limiting the scope of the embodiments. Furthermore, various features of different disclosed embodiments can be combined to form additional embodiments, which are part of this disclosure.

FIG. 1 depicts the re-expansion Experiment Design and embodiments for expanding cells following and IL21 treatment (pre-freeze and well as optionally post-freeze).

FIGS. 2A and 2B depict Population Doubling Level (PDL) comparison between cell expansions with and without IL21 (FIG. 2A) Donor 1, (FIG. 2B) Donor 2.

FIGS. 3A and 3B depict expansion fold Comparison between cell expansions with and without IL21 (FIG. 3A) Donor 1, (FIG. 3B) Donor 2.

FIGS. 4A and 4B depict Population Doubling Level (PDL) comparison between cell Re-stimulation Methods With and Without IL21 (FIG. 4A) Donor 1, (FIG. 4B) Donor 2.

FIGS. 5A and 5B depict expansion fold Comparison between cell Re-stimulation Methods With and Without IL21 (FIG. 5A) Donor 1, (FIG. 5B) Donor 2.

FIG. 6 depicts cytotoxic activity of NK cells against K562 cells expanded with IL-21 (IL-21+).

FIG. 7 depicts cytotoxic activity of NK cells against K562 cells expanded without IL-21 (IL-21−).

FIG. 8 depicts cytotoxic activity of NK cells against K562 cells expanded with IL-21 and re-stimulated with IL-21 (IL-21+/+).

FIG. 9 depicts cytotoxic activity of NK cells against K562 cells expanded with IL-21 and re-stimulated without IL-21(IL-21+/−).

FIG. 10 depicts cytotoxic activity of NK cells against K562 cells expanded without IL-21 and re-stimulated with IL-21 (IL-21−/+).

FIG. 11 depicts cytotoxic activity of NK cells against K562 cells expanded without IL-21 and re-stimulated without IL-21 (IL-21−/−).

FIG. 12A depicts phenotypic comparisons of NK cells' Activating receptors.

FIG. 12B depicts phenotypic comparisons of NK cells' inhibitory and chemokine receptors.

FIG. 13A shows a graph of Population Doubling Level (PDL) of NK cells expanded with IL-21 and re-expanded with IL-21 (IL-21+/+) and NK cells expanded without IL-21 and re-expanded without IL-21 (IL-21−/−).

FIG. 13B shows a graph of Population Doubling Level (PDL) of NK cells expanded with IL-21 and re-expanded with IL-21 (IL-21+/+) and NK cells expanded with IL-21 and re-expanded without IL-21 (IL-21+/−).

FIG. 14A shows a graph of Population Doubling Level (PDL) of NK cells expanded with IL-21 (first stimulation) and re-expanded at least two times (second and third stimulations).

FIG. 14B shows a graph of the corresponding expansion fold of the result in FIG. 14A.

### DETAILED DESCRIPTION

A method for assisting NK cells to go through cryopreservation and recover therefrom has been developed and provided herein. It has been found that CD56+ cells can be successfully expanded after freezing and thawing, if the CD56+ cells are initially (during an initial expansion) co-cultured with feeder cells in the presence of IL-21. By using IL-21, high-purity CD56+ NK cells can result, and surprisingly, they retain an especially large amount of the cytotoxicity. Furthermore, after the NK cells are thawed, they can be further expanded (either without IL-21, or even more advantageously, in the presence of additional IL-21). Thus, IL-21 in a pre-freeze process (such as a first expansion), can allow for superior re-expansion at a later time Also, the resulting product, even after being expanded twice, and frozen once, retains a surprisingly high amount of cytotoxicity, which is enhanced further when IL-21 is not just used during the first expansion, but also used during the second expansion. In some embodiments, this freeze and re-expansion process can be repeated multiple times (each time, optionally with another round of IL-21 during the re-expansion).

In some embodiments, a method of expanding natural killer cells in culture is provided. The method comprises providing PBMCs; co-culturing the PBMCs in the presence of IL-21 for a first period; freezing the co-cultured PBMCs after the first period; thawing the frozen PBMCs; and co-culturing the thawed PBMCs in the presence of IL-21 for a second period. In some embodiments, the PBMC ratio to feeder cells is 1:0.5:0.5. In some embodiments, the ratio can be about 1:0.5:0.5˜1:10:10 for PBMC (e.g., as an alternative to CD56+). In some embodiments, for CD56+ cells: the ratios are multiplied by, for example, 10 or 20 (e.g., 1:1-100 of CD56+ cells to feeder cells). In some embodiments, the expansion is from CD56+ or CD56+/CD3− cells.

According to some embodiments, a method for producing high-purity NK cells can include: co-culturing the cells selected from CD56+ cells and/or CD3−/CD56+ cells together with feeder cells in the presence of a first cytokine (“First Culturing Step” or “First expansion Step”), such as IL-21; freezing the co-cultured cells (“Freezing Step”); thawing the frozen cells (“Thawing Step”); and co-culturing the thawed cells together with added feeder cells (“Second Culturing Step” or “Second Expansion Step”), optionally with more IL-21. Each step is described in greater detail herein. The CD3−/CD56+ cells produced according to the disclosed method can exhibit not only higher purity and higher anti-cancer activity, but also other distinguished characteristics, such as having different surface markers or activated receptors, for example, one or more from CD16, CD25, CD27, CD28, CD69, CD94/NKG2C, CD94/NKG2E, CD266, CD244, NKG2D, KIR2S, KIR3S, Ly94D, NCRs, IFN-a, IFN-b, CXCR3, CXCR4, CX3CR1, CD62L and CD57.

As used herein “step” is part of a process and does not require that one “step” be finished before the next “step” can begin. Unless noted, steps may be provided in overlapping time periods or at the same time, as appropriate. Of course, when one step occurs before an event (such as freezing) and another step occurs after the same event, then there is no overlap (for example, the first IL-21 incubation and the second IL-21 incubation).

In the present specification, the term “CD56+ cells” may be used interchangeably with “CD56+ NK cells”, or “CD56+ natural killer cells”, and the term “CD3−/CD56+ cells” may be used interchangeably with “CD3−/CD56+ NK cells.” The CD56+ cells or CD3−/CD56+ cells can include cells in which CD56 glycoprotein on the cell surface is expressed, or further, cells in which CD3 glycoprotein is not expressed while the CD56 glycoprotein is expressed. Even the same type of immune cells may have differences in CD type attached to the cell surface and expression rate and thus, the functions thereof may be different.

In some embodiments, the CD56+ cells or CD3−/CD56+ cells are obtained by following steps: isolating peripheral blood mononuclear cells (PBMCs) from a blood sample (“First Isolation Step”); isolating cells selected from a group consisting of CD56+ cells and CD3−/CD56+ cells from the peripheral blood mononuclear cells (“Second Isolation Step”).

In the present specification, the “blood sample” may be, but need not be limited to, whole blood of the peripheral blood or leukocytes isolated from the peripheral blood using leukapheresis. Further, the peripheral blood can be obtained from a normal person, a patient having a risk of cancer, or a cancer patient, but the source of the peripheral blood is not limited thereto.

In the present specification, the term “leukapheresis” may refer to a method of selectively removing (isolating) leukocytes from the collected blood and then giving the blood to a patient again, and in some embodiments, the leukocytes isolated by the method may be used without additional methods such as a Ficoll-Hypaque density gradient method.

In the present specification, the term “peripheral blood mononuclear cell” may be used interchangeably with “PBMC”, “mononuclear cell”, and may refer to a mononuclear cell isolated from the peripheral blood which is generally used for anti- cancer immunotherapy. The peripheral blood mononuclear cells may be obtained from the collected human blood using known methods such as a Ficoll-Hypaque density gradient method.

In some embodiments, the peripheral blood mononuclear cells can be autologous, but allogenic peripheral blood mononuclear cells can also be used for producing high-purity NK cells for anti-cancer immunotherapy according to methods described herein. Further, in some embodiments, the peripheral blood mononuclear cells can be obtained from a normal person, but the peripheral blood mononuclear cells can be also obtained from a patient having a risk of cancer and/ or a cancer patient.

In some embodiments, the Second Isolation Step for isolating of the CD56+ natural killer cells from the blood sample can be performed using at least one selected from the group consisting of CD56 microbeads and CD3 microbeads, or an isolating method using equipment such as CliniMACSs, a flow cytometry cell sorter, or MACS Separator, a magnetic sorting system, etc.

For example, the isolating method using the CD56 microbeads and/or the CD3 microbeads can be performed by adding the CD56 microbeads to PBMCs and then removing non-specific binding, or performed by adding the CD3 microbeads to the PBMCs to remove specific binding and then adding the CD56 microbeads again to remove non-specific binding. In some instances, through isolating CD56+ cells and/or CD3−/CD56+ cells from PBMCs, T cells or other non-natural killer cells can be removed.

In the present specification, the term “feeder cell” may refer to a cell that does not divide and proliferate, but has metabolic activity to produce various metabolites and thus, helps the proliferation of target cells.

In some embodiments, the feeder cells can be at least one selected from the group consisting of irradiated Jurkat cells, irradiated Epstein-Barr virus transformed lymphocyte continuous line (EBV-LCL) cells, PBMCs, HFWT, RPMI 1866, Daudi, MM-170, K562 or cells genetically modified by targeting K562 (for example, K562-mbIL-15-41BB ligand). For example, in one embodiment, the feeder cells can be the irradiated Jurkat cells and the EBV-LCL cells. In some embodiments, any feeder cell type can be used, as long as it allows for re-expansion as provided herein, when the NK cells are first expanded in the presence of IL-21 and then frozen, thawed, and then subject to the re-expansion.

In the present specification, the term “Jurkat cell” or “Jurkat cell line” may refer to a blood cancer (immortalized acute T cell leukemia) cell line, which has been developed by Dr. Arthur Weiss of the University of California at San Francisco. Jurkat cells, in which various chemokine receptors are expressed and capable of producing IL-2, have not generally been considered as a possible candidate of the feeder cells for anti-cancer immunotherapy because MHC class I, which is a natural killer cell activation inhibitor, is highly expressed on the cell surface thereof. The Jurkat cells can be obtained from the ATCC (ATCC TIB-152).

In the present specification, the term “EBV-LCL cell” or “EBV-LCL cell line” refers to an Epstein-Barr virus transformed lymphocyte continuous line (EBV-LCL) (D.M. Koelle et al., J Clin Invest, 1993: 91: 961-968), which is a B cell line that is made by infecting human B cells with Epstein-Barr virus in a test tube. The EBV-LCL cells can be directly prepared and used in a general laboratory by a method of adding cyclosporine A in a process of infecting EBV in the PBMC. In some embodiments, the EBV-LCL cell can be prepared by following steps. 30×10⁶ PBMCs are added in 9 mL of a culture medium, the mixture is added in a T 25 culture flask, and then 9 mL of an EBV supernatant is added. 80 μL of cyclosporine A (50 μg/mL) is added and then cultured at 37° C. After 7 days of culture, a half of supernatant is removed, a fresh culture medium is added, and then 40 μL of cyclosporine A is added. The same process can be repeated once every 7 days until 28 days of culture. The cell line can be usable after 28 days of culture, and from this time, the cell line can be cultured in the culture medium without adding cyclosporine A.

The Jurkat cells and the EBV-LCL cells can be used as the feeder cells after irradiation.

In some embodiments, prior to administration, the method can further comprise freezing the expanded cells a second time in a ready-to-inject solution.

In some embodiments, the irradiated Jurkat cells and the irradiated EBV-LCL cells can be included at a content ratio of 1:0.1-5, 1:0.1-4, 1:0.1-3, 1:0.1-2, 1:0.1-1.5, 1:0.5-1.5, 1:0.75-1.25, 0.1-5:1, 0.1-4:1, 0.1-3:1, 0.1-2:1, 0.1-1.5:1, 0.5-1.5:1 or 0.75-1.25:1. For example, the irradiated Jurkat cells and the irradiated EBV-LCL cells can be included at a content ratio of 1:1.

In some embodiments, the irradiated Jurkat cells and the irradiated EBV-LCL cells can be obtained by treating with irradiation of 50-500, 50-400, 50-300, 50-200, 50-150, 70-130, 80-120 or 90-110 Gy. For example, the irradiated Jurkat cells and/or the irradiated EBV-LCL cells can be obtained by treating Jurkat cells and/or EBV-LCL cells with irradiation of 100 Gy.

In the present specification, the term “cytokine” may be used interchangeably with “first cytokine”, or “second cytokine”, and may refer to an immunoactive compound that is usable to induce the peripheral blood mononuclear cells to differentiate into NK cells. In some embodiments, the cytokine is IL-21 (for both the first and the second expansion, and any further rounds of expansion).

As used herein, the terms “co-culture” and “expansion” are interchangeable and denote that the NK cells are being cultured to result in an expanded population of cells. The term “re-expansion” denotes that one round of co-culture or expansion has already occurred for the NK cells. In some embodiments, the co-culture or expansion will occur in the presence of feeder cells and a cytokine, such as IL-21. In some situations herein, the term “culture” is used as a shorthand for “co-culture”.

In some embodiments, the cytokine can be interleukin-2 (IL-2), IL-15, IL-21, FMS-like tyrosine kinase 3 ligand (Flt3-L), a stem cell factor (SCF), IL-7, IL-18, IL-4, type I interferons, a granulocyte-macrophage colony-stimulating factor (GM-CSF), and an insulin-like growth factor 1 (IGF 1), but not limited thereto.

In some embodiments, the first cytokine can be IL-2, IL-21, IL-15, FMS-like tyrosine kinase 3 ligand (Flt3-L), a stem cell factor (SCF), IL-7, IL-18, IL-4, type I interferons, GM-CSF, an insulin-like growth factor 1 (IGF 1), or any combinations thereof. In some embodiments, the second cytokine can be IL-2, IL-21, IL-15, FMS-like tyrosine kinase 3 ligand (Flt3-L), a stem cell factor (SCF), IL-7, IL-18, IL-4, type I interferons, GM-CSF, an insulin-like growth factor 1 (IGF 1), or any combinations thereof. For example, the second cytokine can be IL-21. In some embodiments, more than one cytokine can be present throughout one or more of the steps provided herein. In some embodiments, 11-21 and IL-2 are both present in at least one of the first and second rounds of expansion (or any subsequent rounds thereof). In addition, as various embodiments provided herein also involve the repeated use of cytokines (such as IL-21) in each of the various rounds of expansion, such a cytokine can be employed a first time (or during a first round or step) and a second time (during a re-expansion step for example). Thus, in some embodiments, a first cytokine (such as IL-21) can be used for both the first and second rounds of co-culturing. This can also be alternatively stated, for example, as a first cytokine and a second cytokine, wherein both the first and second cytokines are IL-21.

### Method of Producing NK Cells

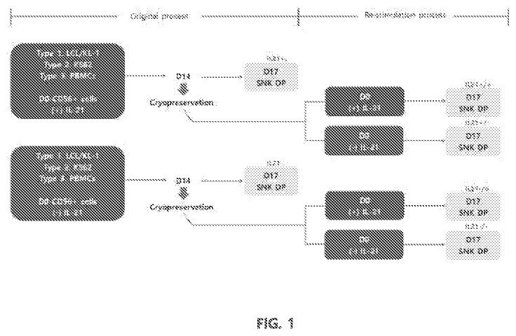

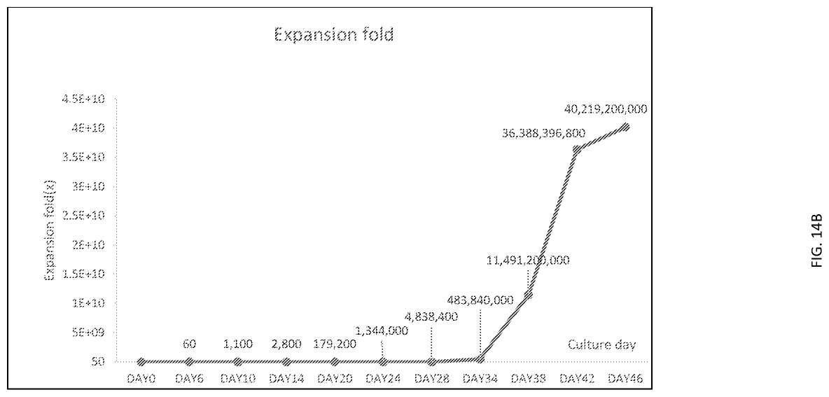

FIG. 1 is a flowchart illustrating some methods of expanding NK cells using various exemplary feeder cells. In some embodiments, the CD56+ cells or CD3−/CD56+ cells are expanded by co-culturing together with feeder cells in the presence of IL-21. The feeder cells can be any type of feeder cell, for example, Jurkat cells and EBV-LCL cells (“Type 1”), K562 cells (“Type 2”) or PBMCs (“Type 3”). In some embodiments, the cells are collected at or about Day 17 (or anywhere in days 16-21 or 9-25) of the culturing, and the produced cells can be referred to as “IL21+.” herein or elsewhere in the specification. Such cell expansion process without cryopreservation or second culturing step can be referred to as “original process” herein or elsewhere in the specification. In some embodiments, the cells are collected at or about at Day 14 (or anywhere in days 14-18 or 9-25) and subjected to cryopreservation. The culturing before the cryopreservation can be referred to as the “first culturing step” or “first expansion step” or “first co-culturing step” herein or elsewhere in the specification. The cryopreserved cells can be thawed and expanded again co-culturing together with feeder cells in the presence of IL-21 (“IL-21+/+”) or in the absence of IL-21 (“IL-21+/−”). Such second expansion process can be referred to as the “second culturing step” or the “re-stimulation process” or the “second co-culturing step” or the second expansion step. The cells can be collected at or about Day 17 (or anywhere in days 16-21 or 9-25) of the culturing. In some embodiments, further re-expansion or re-stimulation steps or cycles can be performed. As shown in FIG. 14B, in some embodiments there can be a first stimulation, followed by two or more re-stimulation steps.

In some embodiments, the CD56+ cells or CD3−/CD56+ cells are expanded by co-culturing together with feeder cells without the presence of IL-21. Typically, this applies at culturing steps after the initial culturing step with IL-21. The feeder cells can be any type of feeder cell for NK cells, including, for example, Jurkat cells and the EBV-LCL cells (“Type 1”), K562 cells (“Type 2”) or PBMCs (“Type 3”). In some embodiments, the cells are collected at or about Day 17 (or days 16-21) of the culturing, and the produced cells can be referred to as “IL21-.” herein or elsewhere in the specification. In some embodiments, the cells are collected at or about at Day 14 (or days 14-18) and subjected to cryopreservation. The cryopreserved cells can be thawed and expanded again co-culturing together with feeder cells in the presence of IL-21 (“IL-21−/+”) or in the absence of IL-21 (“IL-21−/−”). Such second expansion process can be referred to as the “second culturing step” or the “re-stimulation process.” The cells can be collected at or about Day 17 (or days 16-21 or 9-25) of the culturing. As detailed herein, the use of IL-21 in the first expansion allows for freezing and thawing and subsequent superior expansion of NK cells, optionally with additional IL-21 (which has even further benefits of improved cytotoxicity, for example).

### First Culturing (Expansion or Co-Culture) Step

The first culturing step can include adding the cytokine once or more between day 0-6 of culturing. More than one cytokine can be used (for example IL-2 can also be employed). For example, the first culturing step can include adding one or both of the cytokines once on each of day 0 and day 3 of culturing.

When co-culturing with feeder cells and the first cytokine, culturing with further addition of another cytokine once or more during day 0-6 can exhibit superior proliferation and/or anti-cancer activity. In some embodiments, culturing with the addition of the feeder cells and the additional cytokine for six days in the cycle of 14 days can exhibit superior proliferation and/or anti-cancer activity. In some embodiments, IL-21 is used at least once, and optionally pre and post freeze. In some embodiments IL-2 can be included as a further cytokine.

In some embodiments, the first cytokine (for example, IL-21) can be used at a concentration of 10-1,000, 10-500, 10-100, 20-100, 30-100, 40-100, 50-100, or 10-50 ng/mL. In some embodiments, the additional cytokine can be used at a concentration of 50-1,000, 50-900, 50-800, 50-700, 50-600, 50-550, 100-550, 150-550, 200-550, 250-550, 300-550, 350-550, 400-550, or 450-550 IU/mL. In some embodiments, the concentration is about 50 ng/ml.

Conventional methods of proliferating NK cells utilize high concentrations of various cytokines. Conversely, in some embodiments of the method of proliferating NK cells described herein, NK cells with high yield and high purity can be proliferated using only low concentrations of one cytokine.

In some embodiments, the co-culturing (culturing, expansion (including re-expansion)) can be performed by including the peripheral blood mononuclear cells and the feeder cells (for example, the Jurkat cells and the EBV-LCL cells) at a mixing ratio of 1:1-100, 1:1-90, 1:1-80, 1:1-70, 1:10-65, 1:20-65, 1:30-65, 1:40-65, 1:50-65 or 1:55-65. In some embodiments, the co-culturing can be performed by including the peripheral blood mononuclear cells and the feeder cells (for example, the Jurkat cells and the EBV-LCL cells) at various mixing ratios. In some embodiments, the ratio can be about 1:0.5:0.5˜1:10:10 for PBMC (e.g., as an alternative to CD56+). In some embodiments, for CD56+ cells: the ratios are multiplied by for example 10 or 20 (e.g., 1:1-100 of CD56+ cells to feeder cells).

The co-culturing can be performed in a medium and any suitable media generally used for induction and proliferation of the peripheral blood mononuclear cells to the NK cells in the art can be used without a limitation as such a medium. For example, an RPMI-1640, DMEM, x-vivo10, x-vivo20, or cellgro SCGM medium can be used as such a medium. In addition, the culture conditions such as a temperature can follow any suitable culture conditions of the peripheral blood mononuclear cells known in the art.

In some embodiments, the first culturing step can be performed for 0-45, 0-42, 0-40, 0-30, 0-20, 0-19, 0-18, 0-17, 0-16, 0-15 or 0-14 days.

### Freezing Step

The natural killer cells cultured and provided from the first culturing step can be collected and suspended in a medium, and subsequently frozen and cryopreserved. In some embodiments, the medium can include FBS and/or DMSO. For example, the medium can include 90% FBS and 10% DMSO, or 90-95% FBS and 5-10% DMSO. In some embodiments, other acceptable cryo-preservatives such as a CryoStor solution (CS10, CS5) etc. or other components such as sucrose or glycerol can be included. In some embodiments, suitable preservatives include DMSO, glycerol, ethylene glycol, sucrose, trehalose, dextrose, polyvinylpyrrolidone, or the like. In some embodiments, IL-2 can be present and/or Human Serum Albumin.

In some embodiments, cryopreservation can include transferring provided natural killer cells into cryopreservation container with isopropyl alcohol, freezing the natural killer cells in the cryopreservation container in a ultra-low freezer overnight, and preserving the natural killer cells at −192° C. or lower. In some embodiments, the frozen natural killer cells can be preserved as frozen at −10° C. or lower, −20° C. or lower, −50° C. or lower, −70° C. or lower, −100° C. or lower, −150° C. or lower, −192° C. or lower, or −200° C. or lower. In some embodiments, the frozen natural killer cells can be preserved for a day or more, 2 days or more, 3 days or more, 7 days or more, 14 days or more, 30 days or more, 60 days or more, or 180 days or more, including any range between any two of the preceding values. In some embodiments, the temperature is from −135 C to −196 C. In some embodiments, the cells are stored for 0.5, 1, 2, 3, 4, or 5 years, including any range between any two of the preceding values.

In some embodiments, the provided natural killer cells can be cooled and/or frozen using a controlled rate freezer (CRF). In some embodiments, the frozen natural killer cells can be preserved under liquid nitrogen.

In some embodiments, the provided natural killer cells can be cooled and/or frozen using a controlled rate freezer (CRF). In some embodiments, this can be done at a slow rate (e.g., 1-8 hours, e.g., 1, 2, 3, 4, 5, 6, 7 or 8 hours or longer). It can also be slowly frozen manually by using Isopropyl alcohol, in which. Vials of NK cells are placed in cryo-containers (e.g. Nalgene Mr. Frosty), and store at −70° C. overnight. The next day the cells are transferred to liquid nitrogen (LN₂).

### Thawing Step

The frozen natural killer cells can be thaw after cryopreservation using any suitable methods. In some embodiments, the frozen/cryopreserved natural killer cell can be thawed using water bath, for example at 37° C. In some embodiments, the frozen natural killer cell can be thawed for an hour or more, two hours or more, five hours or more, or ten hours. In some embodiments, the process is conducted in a water or bead bath. In some embodiments, the thawing process is done as soon as possible or immediately after taking from the frozen state (e.g., the liquid nitrogen).

In some embodiments, the frozen Natural Killer Cells can be thawed within 10 minutes in a 37 C water bath, in which the frozen vial or bag can be shaken as to accelerate the thawing process. In some embodiments, the cells can also be thawed using an instrument such as a Heat block, an automated cell thawing instrument for vials (e.g. ThawStar, Biocision), or a thawing instrument for bags (e.g., VIA Thaw, GE healthcare).

### Second Culturing (Second Co-culture, Second Expansion, or Re-expansion, or Any Subsequent Culturing Step) Step

Because the cells are initially processed with IL-21 in the first culturing step, a second expansion step thereby becomes possible. Thus, the method can include not just one expansion step, but two expansion steps (e.g., one or more re-expansion steps). Preferably, the second expansion step occurs after the sample has been frozen, stored for some period of time, and then thawed.

During the second culturing step, the thawed natural killer cells can be cultured with the addition of feeder cells for once or more times.

In some embodiments, the feeder cells can be added once or more during a 14 day cycle (or 9-25 day cycle) of culturing.

In some embodiments, culturing with the addition of the feeder cells once or more during a 14 day cycle can not only exhibit superior proliferation and/or anti-cancer activity, but also maintain sustained cell growth after freezing and thawing, such that natural killer cells are produced in enough quantity for clinical use.

In some embodiments, the second culturing step can include adding the second round of cytokine (e.g., additional IL-21 or other cytokines in addition to the IL-21).

In some embodiments, the second culturing step can include adding the subsequent round of cytokine once of more during day 0-6 of culturing.

Any description of the cytokines elsewhere herein can apply to the cytokines for the second culturing step. For example, in some embodiments, the second cytokine can be used at a concentration of 10-1,000, 10-500, 10-100, 20-100, 30-100, 40-100, 50-100, or 10-50 ng/mL and/or the additional cytokine can be used at a concentration of 50-1,000, 50-900, 50-800, 50-700, 50-600, 50-550, 100-550, 150-550, 200-550, 250-550, 300-550, 350-550, 400-550, or 450-550 IU/mL. The cytokine used for the second expansion is preferably IL-21.

In some embodiments, the composition is one of frozen cells before re-expansion, which can include: IL-2; 5-10% DMSO; 90-95% FBS; and NK cells that are optionally CD56+ cells. In some embodiments, the composition is frozen solid. In some embodiments, the NK cells are at least 90% of a cell population of the composition. In some embodiments, it further comprises CryoStor solution. In some embodiments, the composition is for frozen cells before re-expansion.

In some embodiments, the composition is one for frozen cells before re-expansion, which can include: IL-2; 5-10% DMSO; 80-95% Hartman solution; 1-10% human serum albumin; and NK Cells. In some embodiments, it further comprises CryoStor solution. In some embodiments, the composition is for frozen cells before injection.

In some embodiments, a method of expanding natural killer cells in culture can include isolating CD56+ cells from a blood sample; co-culturing the isolated CD56+ cells in the presence of IL-21 for a first period; freezing the co-cultured CD56+ cell after the first period; thawing the frozen CD56+ cells; and co-culturing the thawed CD56+ cells in the presence of IL-21 for a second period.

In some embodiments, the method can further include storing the frozen CD56+ cells at a temperature lower than −100° C. In some embodiments, the frozen CD56+ cells may be stored at a temperature of −10° C. or lower, −20° C. or lower, −50° C. or lower, −70° C. or lower, −150° C. or lower, −192° C. or lower, or −200° C. or lower. In some embodiments, the frozen CD56+ cells can be stored for more than a day before thawing. In some embodiments, the frozen CD56+ cells can be stored for 2 days or more, 3 days or more, 7 days or more, 14 days or more, 30 days or more, 60 days or more, or 180 days or more, including any range between any two of the preceding values. In some embodiments, the cells can be frozen for as long as they are viable when thawed.

In some embodiments, the isolated CD56+ cells can be co-cultured for between 13-16 days before freezing. For example, the isolated CD56+ cells can be co-cultured for 14 or 15 days before freezing. In some embodiments, the co-culture or expansion can go for any time as is appropriate. In some embodiments, the co-culture or expansion (including re-expansion) can be for 9-25 days, e.g., 10-24, 11-23, 13-22, 14-21, 14-18, 14-16 days, etc. These time frames can be applied to any of the expansion and/or re-expansion periods provided herein (including for embodiments to other cells).

In some embodiments, the isolated CD56+ cells can be co-cultured with one or more irradiated feeder cells in the presence of IL-21. In some embodiments, the thawed CD56+ cells can be co-cultured with one or more irradiated feeder cells in the presence of IL-21. The one or more feeder cells can include, but are not limited to one or more selected from a group consisting of irradiated Jurkat cells, irradiated Epstein-Barr virus transformed lymphocyte continuous line (EBV-LCL) cells, K562 cells, mb15-k562, mb21-k562 feeder cells, HuT78, and/or PBMCs. In some embodiments, expansion is done with PBMC, CD56+ and/or CD56+CD3− cells. In some embodiments, the CD56+ cells can be co-cultured with a ratio of about 1:1-100 of CD56+ cells to feeder cells. For example, the CD56+ cells can be co-cultured with a ratio of about 1:2, 1:5, 1:10, 1:30 or 1:100 of CD56+ cells to feeder cells.

In some embodiments, IL-21 can be added at a concentration of 10-100 ng/mL during the first and/or second period. For example, IL-21 can be added at a concentration of 20-80 ng/mL, 30-70 ng/mL, or 40-60 ng/mL during the first and/or second period. In some embodiments, IL-21 can be added more than once during the first and/or second period.

In some embodiments, a method of expanding natural killer cells in culture can include isolating CD56+ from a blood sample; co-culturing the CD56+ cells with one or more feeder cells in the presence of IL-21; freezing the CD56+ cells; thawing the frozen CD56+ cells; and expanding the thawed CD56+ cells.

In some embodiments, the CD56+ cells can be frozen at a temperature lower than −100° C. In some embodiments the CD56+ cells can be frozen at a temperature of −10° C. or lower, −20° C. or lower, −50° C. or lower, −70° C. or lower, −150° C. or lower, −192° C. or lower, or −200° C. or lower. In some embodiments, the frozen CD56+ cells can be stored for more than a day before thawing. In some embodiments, the frozen CD56+ cells can be stored for 2 days or more, 3 days or more, 7 days or more, 14 days or more, 30 days or more, 60 days or more, or 180 days or more, including any range between any two of the preceding values. For example, the frozen CD56+ cells may be stored for a period more than a day and less than 10 years.

In some embodiments, the CD56+ cells can be co-cultured for between 13-16 days before freezing. For example, the CD56+ cells can be co-cultured for 14 or 15 days before freezing. In some embodiments, the co-culture or expansion (including re-expansion) can be for 9-25 days, e.g., 10-24, 11-23, 13-22, 14-21, 14-18, 14-16 days, etc. These time frames can be applied to any of the expansion and/or re-expansion periods provided herein (including for embodiments to other cells).

In some embodiments, the one or more feeder cells can be one or more selected from a group consisting of irradiated Jurkat cells, irradiated Epstein-Barr virus transformed lymphocyte continuous line (EBV-LCL) cells, K562 cells and PBMCs. The CD56+ cells can be co-cultured with a ratio of about 1:1-100 of CD56+ cells to feeder cells. For example, the CD56+ cells can be co-cultured with a ratio of about 1:2, 1:5, 1:10, 1:30 or 1:100 of CD56+ cells to feeder cells.

In some embodiments, IL-21 can be added at a concentration of 10-100 ng/mL. For example, IL-21 can be added at a concentration of 20-80 ng/mL, 30-70 ng/mL, or 40-60 ng/mL. In some embodiments, IL-21 can be added more than once.

In some embodiments, a method of increasing cytotoxicity of natural killer cells can include providing said natural killer cells; freezing said natural killer cells; thawing the frozen natural killer cells; and co-culturing the thawed natural killer cells with one or more feeder cells in the presence of IL-21.

In some embodiments, the method can further include storing the frozen natural killer cells at a temperature lower than −100° C. In some embodiments, the frozen natural killer cells may be stored at a temperature of −10° C. or lower, −20° C. or lower, −50° C. or lower, −70° C. or lower, −150° C. or lower, −192° C. or lower, or −200° C. or lower. In some embodiments, the frozen natural killer cells can be stored for more than a day before thawing. In some embodiments, the frozen natural killer cells can be stored for 2 days or more, 3 days or more, 7 days or more, 14 days or more, 30 days or more, 60 days or more, or 180 days or more, including any range between any two of the preceding values. In some embodiments, the cells are stored for as long as any of the cells remain viable once thawed.

In some embodiments, the one or more feeder cells can be one or more selected from a group consisting of irradiated Jurkat cells, irradiated Epstein-Barr virus transformed lymphocyte continuous line (EBV-LCL) cells, K562 cells and PBMCs. In some embodiments, the thawed natural killer cells can be co-cultured with a ratio of about 1:1-100 of the natural killer cells to the feeder cells.

In some embodiments, IL-21 can be added at a concentration of 10-100 ng/mL. For example, IL-21 can be added at a concentration of 20-80 ng/mL, 30-70 ng/mL, or 40-60 ng/mL. In some embodiments, IL-21 can be added more than once.

In some embodiments, a method for producing natural killer cells can include repeating following steps: the freezing step; the thawing step; and the second culturing step including co-culturing with addition of the feeder cells.

In some embodiments, as shown in FIG. 14A (in the bar time points above the data curve), more than one cycle of re-stimulation or re-expansion can be applied. In some embodiments, there is a first stimulation, followed by cell culturing, followed by freezing (optional), followed by a second stimulation (a re-stimulation) followed by a second cell culture step, followed by freezing (optional), followed by a third stimulation (a second re-stimulation) followed by another culture step. IL-21 can be used in each of the stimulation steps as provided herein. The optional freezing step can be applied to the entirety of the cells, or a fraction of the cells. In some embodiments, there are 2, 3, 4, 5, 6, 7, 8, 9, 10 or more rounds of stimulation (e.g., one round of stimulation and then 1, 2, 3, 4, 5, 6, 7, 8, 9 or more rounds of re-stimulation). Following each of the re-stimulations, there can be another cell culture/expansion step. In some embodiments, each of the cell culture or re-expansions can go for 9-25 days. There can be a freeze step following each cell culture step. In some embodiments, the process can be a cycle of: a) stimulation (or re-stimulation), followed by b) cell culture, followed by c) freezing (optional), followed by d) thawing (optional), to be repeated as many times as desired. In some embodiments, the amount of IL-21 is between 10 and 100 ng/mL, e.g., 50 ng/mL. The stimulation/restimulation in FIG. 14A denotes the addition of IL-21 to the cells.

In some embodiments, any of the processes provided herein can include one or more freezing step.

In some embodiments, any of the embodiments regarding NK cells provided herein can include natural NK cells as well as genetically modified NK cells.

### Cell Therapeutic Composition for Treating Cancer

According to some embodiments, a cell therapeutic composition for the treatment of cancer can include peripheral blood derived CD56+ NK cells. The cells will have gone through or been the result of at least two rounds of expansion, at least the first round of which is in the presence of IL-21.

In the present specification, the term “peripheral blood-derived” can mean that the cells are derived from “whole blood of the peripheral blood” or “leukocytes isolated from the peripheral blood using leukapheresis.” The peripheral blood derived CD56+ NK cells can be used interchangeably with peripheral blood mononuclear cell (PBMC) derived CD56+ NK cells.

In some embodiments, the cytokine can be used at a concentration of 18-180,000, 20-100,000, 50-50,000, 50-1,000, 50-900, 50-800, 50-700, 50-600, 50-550, 100-550, 150-550, 200-550, 250-550, 300-550, 350-550, 400-550, 450-550 IU/mL. When the cytokine is used in these ranges, it can suppress apoptosis of the NK cells included in the cancer treatment composition and increase anti-cancer activity of the NK cells.

In some embodiments, the composition can include IL-2 as an additional cytokine (e.g., in addition to IL-21).

In some embodiments, the CD56+ NK cells can be obtained as described elsewhere herein. For example, the CD56+ NK cells can be obtained by coculturing with feeder cells (e.g. irradiated Jurkat cells and irradiated EBV-LCL cells). In some embodiments, the ratio of CD56+ NK cells to whole cells (purity) can be 85% or more, 90% or more, 95% or more, or 98% or more.

In some embodiments, the cancer can be blood cancer, stomach cancer, pancreatic cancer, cholangiocarcinoma, colon cancer, breast cancer, liver cancer, ovarian cancer, lung cancer, kidney cancer, prostate cancer or neuroblastoma, but not limited thereto. In some embodiments, the process can be applied to allogenic NK cell Therapy in, for example, neurodegenerative disease and acute infection.

In some embodiments, the composition may not include T cells, or may include only trace amount of T cells. For example, the ratio of T cells to whole cells in the composition can be less than 15%, less than 10%, less than 5%, less than 2%, less than 1% or less.

In the present specification, the term “T cell” refers to a lymphocyte derived from thymus, which can “memorize” previously encountered antigens and provide information to B cells, thereby facilitates production of antibody and plays an important role in cell immune system. Since these T cells can distinguish very small differences among different antigens to induce an immune response to allogenic antigens, autologous therapy is possible, but there can be a limit to be used for allogenic therapy. Accordingly, the cell therapeutic composition without T cells can be suitable for allotransplantation.

In the present specification, the term “cell therapeutic agent” refers to a medicine which is used for treatment, diagnosis, and prevention through a series of actions, such as proliferating and screening autologous, allogenic, and xenogenic living cells in vitro for restoring functions of cells and tissues or changing biological characteristics of the cells by other methods. The cell therapeutic agents have been regulated as medical products from 1993 in USA and 2002 in Korea. These cell therapeutic agents can be largely classified into two fields, that are, first, stem cell therapeutic agents for tissue regeneration or recovery of organ functions, and second, immune cell therapeutic agents for regulation of immune responses, such as inhibition of the immune response or enhancement of the immune response in vivo.

An administration route of cell therapeutic compositions described herein can be any suitable route as long as the composition reaches a target tissue. The administration can be parenteral administration, for example, intraperitoneal administration, intravenous administration, intramuscular administration, subcutaneous administration, or intradermal administration, but not limited thereto.

The cell therapeutic composition described herein can be formulated in a suitable form together with a pharmaceutically acceptable carrier suitable or generally used for cell therapy. The “pharmaceutically acceptable” refers to a composition which is physiologically acceptable and does not generally cause an allergic reaction such as gastrointestinal disorders, dizziness, or the like, or similar reactions thereto, when being administered to the human body. The pharmaceutically acceptable carrier can include, for example, parenteral administration carries such as water, suitable oils, saline, aqueous glucose and glycol, and the like, and further include stabilizers and preservatives. The suitable stabilizer includes an antioxidant such as sodium hydrogen sulfite, sodium sulfite, or ascorbic acid, sucrose, albumin, or the like. The suitable preservative includes DMSO, glycerol, ethylene glycol, sucrose, trehalose, dextrose, polyvinylpyrrolidone, or the like.

The cell therapeutic composition can also be administered by any device in which the cell therapeutic agent can move to the target cell.

The cell therapeutic composition can include a therapeutically effective amount of cell therapeutic agent for treatment of diseases. The term “therapeutically effective amount” means an amount of an active ingredient or a cell therapeutic composition which induces biological or medical responses in tissue systems, animals, or humans which are considered by researchers, veterinarians, physicians, or other clinicians, and includes an amount of inducing alleviation of symptoms of diseases or disorders to be treated. It will be apparent to those skilled in the art that the cell therapeutic agent included in the cell therapeutic composition can be changed according to a desired effect. Therefore, the optimal content of the cell therapeutic agent can be easily determined by those skilled in the art, and can be adjusted according to various factors including a type of disease, severity of the disease, contents of other ingredients contained in the composition, a type of formulation, and an age, a weight, a general health condition, a gender, and a diet of a patient, an administration time, an administration route, a secretion ratio of the composition, a treatment period, and simultaneously used drugs. It is important to include an amount capable of obtaining a maximum effect by a minimum amount without side effects by considering all of the factors. For example, the cell therapeutic composition can include a cell therapeutic agent of 1×10⁶ to 5×10⁸ cells per kg of body weight.

In some embodiments, the NK cells of the cell therapeutic composition can have cytotoxicity 50% or greater, 60% or greater, 70% or greater, 80% or greater, 85% or greater, 90% or greater, 93% or greater, 95% or greater, or 98% or greater compared to their pre-frozen population.

In some embodiments, a composition can include an effective amount of CD56+ cells derived from peripheral blood mononuclear cells (PBMCs) from the patient. The CD56+ cells can be prepared by isolating peripheral blood mononuclear cells (PBMCs) from a blood sample; isolating CD56+ cells from the PBMCs; co-culturing the CD56+ cells with one or more feeder cells in the presence of one or more cytokines; freezing the CD56+ cells; thawing the frozen CD56+ cells; and co-culturing the thawed CD56+ cells with one or more feeder cells in the presence of one or more cytokines.

In some embodiments, the effective amount of CD56+ cells can be 1×10⁶ to 5×10⁸ cells per kg of body weight.

In some embodiments, a cell composition can include an effective amount of CD56+ cells derived from peripheral blood mononuclear cells (PBMCs) from the patient; IL-2; and IL-21.

In some embodiments, a composition can include a first population of CD56+ cells derived from peripheral blood mononuclear cells (PBMCs); ice; IL-2 and IL-21. When thawed, the CD56+ cell has a cytotoxicity of at least 80% of a second population of CD56+ cells, wherein the second population of CD56+ cells have not been frozen. In some embodiments, the cytotoxicity is at least 85, 90, 95, 96, 97, 98, or 99%.

In some embodiments, comparing a non-frozen expansion (with and without IL21, IL21+or IL21−) versus one that was frozen but co cultured with IL21+ on the first step, the average cytotoxicity of the frozen expansion was 97.7% (Table C: Range 83%-131%) of the non-frozen. In some embodiments, when comparing a non-frozen expansion (with and without IL21, IL21+or IL21−) versus one that was frozen but not co cultured with IL21+on the first step (IL21−−, IL21−+), the average cytotoxicity of the frozen expansion can be at least 81.4% of the non-frozen expansion (table D Range 61%-83%). When IL21 was added in both steps of the frozen expansion (IL21++), the average cytotoxicity was 114% of the non-frozen expansion (Table C values 98%-131%). Thus, in some embodiments, IL21+/+(72%) vs IL21−/+(45%) it denotes that when first expanding with IL21 and also on the second step the cytotoxicity is 60% higher than without Il21 on the first step. Thus, in some embodiments, the presence of IL21 in the first expansion allows for a second, post freeze expansion that is 60% higher. For IL21+/− (62%) vs IL21 −/− (46.1%) it means that when first expanding with IL21 on the first step but not on the second step, the cytotoxicity can be at least 35% higher than without Il21 on the first step.

In some embodiments, the cytotoxicity is comparing a non-frozen expansion (with and without IL21) versus one that was frozen but co cultured with IL21+ on the first step, wherein an average cytotoxicity of the frozen expansion is 97.7% of the non-frozen. In some embodiments, the cytotoxicity is comparing a non-frozen expansion (with and without IL21) versus one that was frozen but not co cultured with IL21+ on the first step, wherein an average cytotoxicity of the frozen expansion is 81.4% of the non-frozen expansion. In some embodiments, where, IL21 is added both before and after freezing, and wherein an average cytotoxicity is 114% of a non-frozen expansion.

In some embodiments, the superior nature of the initial IL21 treatment during co-expansion to allow for subsequent re-expansion can be in line with the results in the following tables A-D:

TABLE A
Cytotoxicity k562 % Lysis

| | 10:1 | 3:1 | 1:1 | 0.5:1 |
|---|---|---|---|---|
| IL21+ | 95.8 | 98.8 | 74.6 | 53.3 |
| IL21− | 94.2 | 81.4 | 55.6 | 37.7 |
| IL21+/+ | 98.1 | 84.9 | 72.8 | 42.9 |
| IL21+/− | 99.6 | 83.2 | 62.2 | 33.6 |
| IL21−/+ | 89.1 | 81 | 45.3 | 31.2 |
| IL21−/− | 91.6 | 77.3 | 46.1 | 30.8 |

TABLE B
Comparison with IL21 in first step

| | | 10:1 | 3:1 | 1:1 | 0.5:1 | AVG |
|---|---|---|---|---|---|---|
| 2-step frozen/ | IL21++/IL21−− | 107% | 110% | 158% | 139% | 129% |
| 2-step frozen | | | | | | |
| 2-step frozen/ | IL21++/IL21+− | 98% | 102% | 117% | 128% | 111% |
| 2-step frozen | | | | | | |
| 2-step frozen/ | IL21++/IL21−+ | 110% | 105% | 161% | 138% | 128% |
| 2-step frozen | | | | | | |
| 2-step frozen/ | IL21+−/IL21−− | 109% | 108% | 135% | 109% | 115% |
| 2-step frozen | | | | | | |

TABLE C
Comparison with IL21 in first step

| | | 10:1 | 3:1 | 1:1 | 0.5:1 | AVG |
|---|---|---|---|---|---|---|
| 2-step frozen/ | IL21++/ IL21+ | 102% | 86% | 98% | 80% | 92% |
| 1-step Unfrozen | | | | | | |
| 2-step frozen/ | IL21++/IL21− | 104% | 104% | 131% | 114% | 113% |
| 1-step Unfrozen | | | | | | |
| 2-step frozen/ | IL21+−/IL21+ | 104% | 84% | 83% | 63% | 84% |
| 1-step Unfrozen | | | | | | |
| 2-step frozen/ | IL21+−/IL21− | 106% | 102% | 112% | 89% | 102% |
| 1-step Unfrozen | | | | | | |
| | Total Average: | 97.7% | | | | |

TABLE D
Comparison without IL21 in first step

| | | 10:1 | 3:1 | 1:1 | 0.5:1 | AVG |
|---|---|---|---|---|---|---|
| 2-step frozen/ | IL21−−/IL21− | 97% | 95% | 83% | 82% | 89% |
| 1-step Unfrozen | | | | | | |
| 2-step frozen/ | IL21−−/IL21+ | 96% | 78% | 62% | 58% | 73% |
| 1-step Unfrozen | | | | | | |
| 2-step frozen/ | IL21−+/IL21− | 95% | 100% | 81% | 83% | 90% |
| 1-step Unfrozen | | | | | | |
| 2-step frozen/ | IL21−+/IL21+ | 93% | 82% | 61% | 59% | 74% |
| 1-step Unfrozen | | | | | | |
| | Total Average: | 81.4% | | | | |

In any of the embodiments for culturing, expanding (including re- expanding) NK cells, the number of peripheral blood mononuclear cells in the culture at the start of the co-culturing (culturing, expansion (including re-expansion)) is in a range of 1×10⁴ to 1×10¹⁵ cells. In some embodiments, the number of peripheral blood mononuclear cells in the culture at the start of the co-culturing is in a range of 1×10⁴ to 5×10⁴ cells, 5×10⁴ to 1×10⁵ cells, 1×10⁵ to 5×10⁵ cells, 5×10⁵ to 1×10⁶ cells, 1×10⁶ to 1×10⁷ cells, 1×10⁷ to 1×10⁸ cells, 1×10⁸ to 1×10⁹ cells, 1×10⁹ to 1×10¹⁰ cells, 1×10¹¹ to 1×10¹² cells, 1×10¹² to 1×10¹³ cells, 1×10¹³ to 1×10¹⁴ cells, or 1×10¹⁴ to 1×10¹⁵ cells. In some embodiments, the number of peripheral blood mononuclear cells in the culture at the start of the first or initial expansion is in a range of 1×10⁴ to 5×10⁴ cells, 5×10⁴ to 1×10⁵ cells, 1×10⁵ to 5×10⁵ cells, 5×10⁵ to 1×10⁶ cells, 1×10⁶ to 1×10⁷ cells, 1×10⁷ to 1×10⁸ cells, 1×10⁸ to 1×10⁹ cells, 1×10⁹ to 1×10¹⁰ cells, 1×10¹¹ to 1×10¹² cells, 1×10¹² to 1×10¹³ cells, 1×10¹³ to 1×10¹⁴ cells, or 1×10¹⁴ to 1×10¹⁵ cells. In some embodiments, the number of peripheral blood mononuclear cells in the culture at the start of the re-expansion is in a range of 1×10⁴ to 5×10⁴ cells, 5×10⁴ to 1×10⁵ cells, 1×10⁵ to 5×10⁵ cells, 5×10⁵ to 1×10⁶ cells, 1×10⁶ to 1×10⁷ cells, 1×10⁷ to 1×10⁸ cells, 1×10⁸ to 1×10⁹ cells, 1×10⁹ to 1×10¹⁰ cells, 1×10¹¹ to 1×10¹² cells, 1×10¹² to 1×10¹³ cells, 1×10¹³ to 1×10¹⁴ cells, or 1×10¹⁴ to 1×10¹⁵ cells. The present methods can provide for greater expansion of NK cells than conventional approaches. Thus, in any of the above embodiments, the number of peripheral blood mononuclear cells in the culture at the start of the co-culturing (culturing, expansion (including re-expansion)) is a number of cells that would be difficult to use in a conventional approach for expanding NK cells (e.g., expanding without cytokines such as IL-21 and/or IL-2) to provide a similar number of NK cells, e.g., for therapeutic use and/or cryopreservation.

As disclosed herein, methods of the present disclosure in some embodiments provide for NK cells suitable for therapeutic use, e.g., for immunotherapy. In some embodiments, methods of the present disclosure provide for cryopreservation of NK cells that can be subsequently thawed and expanded effectively for therapeutic use. Thus, in any of the embodiments for culturing or expanding (including re-expanding) NK cells, the NK cells are expanded to produce one population of expanded NK cells for therapeutic use, and another population for cryopreservation. In some embodiments, the cryopreserved NK cells are later thawed and re-expanded for therapeutic use and/or further cryopreservation.

In any of the embodiments for culturing or expanding (including re-expanding) NK cells, the number of NK cells at the end of expansion or re-expansion is greater than the number of NK cells that is effective for therapeutic use. In some embodiments, the excess NK cells are cryopreserved for future use, e.g., future thawing, expansion and administration to a patient in need thereof. In some embodiments, NK cells are expanded or re-expanded to an extent at least 10%, 20%, 30%, 40%, 50%, 60%, 70%, 80%, 90%, 100%, 150%, 200%, or more, or a percentage in a range between any two of the preceding values, greater in cell number than the number of NK cells used or to be used for therapy, e.g., immunotherapy.

In any of the embodiments for culturing or expanding (including re-expanding) NK cells, the method can include repeating the freeze-thaw-expansion cycle. In some embodiments, the method includes repeating the freeze-thaw-expansion cycle one, two, three, four, five, six, seven, eight or more times.

Also provided herein is a composition of cryopreserved NK cells, where the NK cells retain their biological activity, e.g., cytotoxicity, after thawing. In some embodiments, the cryopreserved NK cells retain their biological activity, e.g., cytotoxicity, after thawing and re-expanding. In some embodiments, the composition of cryopreserved NK cells is prepared by any of the methods for culturing or expanding (including re-expanding) NK cells, as disclosed herein. In some embodiments, the composition includes a population of immune cells that is at least 80%, 85%, 90%, 95%, 97% or more NK cells. The composition can contain a suitable cryopreservation medium. In some embodiments, the composition includes dimethylsulfoxide (DMSO) and serum (e.g., FBS, human serum). In some embodiments, the composition includes DMSO at 1-15%, 2-15%, 5-15%, 5-10%, or about 10%. In some embodiments, the composition consists or comprises of a population of cryopreserved immune cells including at least 90% NK cells, 10% DMSO and 90% FBS. In some embodiments, the NK cells are derived from PBMC obtained from a subject. In some embodiments, the composition consists or comprises of a population of cryopreserved immune cells including at least 90% NK cells, 5-10% DMSO and 90-95% FBS. In some embodiments, the NK cells are derived from PBMC obtained from a subject. In some embodiments, it further comprises CryoStor solution.

### Method for Preventing or Treating Cancer

In some embodiments, a method for preventing or treating cancer is provided. The method comprises administering a cell therapeutic composition for anti-cancer including peripheral blood-derived CD56+ natural killer cells and cytokines to a subject. In some embodiments, the cell is the result of a two-fold expansion process, at least the first of which occurs in the presence of IL-21.

The term “subject” refers to a mammal which is a subject for treatment, observation, or testing, and preferably, a human The subject can be a patient of blood cancer, stomach cancer, pancreatic cancer, cholangiocarcinoma, colon cancer, breast cancer, liver cancer, ovarian cancer, lung cancer, kidney cancer, prostate cancer or neuroblastoma, but not limited thereto.

In some embodiments, in the case of an adult, the cell therapeutic composition can be administered once to several times a day. The cell therapeutic composition can be administered every day or in a 2-180 day interval. the cell therapeutic agent included in the composition can include 1×10⁶ to 1×10¹¹ peripheral blood-derived CD56+ natural killer cells, for example, about 1×10⁶ to 1×10⁸ NK cells per kg of body weight. In some embodiments, the peripheral blood-derived CD56+ natural killer cells in the cell therapeutic composition are at least about 90% pure. In some embodiments, the cytokine is IL-2 at a concentration ranging from about 50-50,000 IU/ml.

In some embodiments, the cell therapeutic composition can be formulated in a suitable form together with a pharmaceutically acceptable carrier suitable or generally used for cell therapy. The “pharmaceutically acceptable” refers to a composition which is physiologically acceptable and does not generally cause an allergic reaction such as gastrointestinal disorders, dizziness, or the like, or similar reactions thereto, when being administered to the human body. The pharmaceutically acceptable carrier can include, for example, parenteral administration carriers such as water, suitable oils, saline, aqueous glucose, glycol, base compounds such as Hartman solution, or alternatives like normal saline solution, plasmalyte A and the like, and further include stabilizers and preservatives. The suitable stabilizer includes an antioxidant such as sodium hydrogen sulfite, sodium sulfite, or ascorbic acid, sucrose, albumin, human serum albumin or the like. The suitable preservative includes DMSO, glycerol, ethylene glycol, sucrose, trehalose, dextrose, polyvinylpyrrolidone, or the like.

In some embodiments, the cell therapeutic composition can be administered by any suitable method, such as administration through a rectal, intravenous, intraarterial, intraperitoneal, intramuscular, intrasternal, percutaneous, topical, intraocular, or intradermal route. In some embodiments, the NK cells included in the composition can be allogenic, i.e. obtained from a person other than the subject being treated. In some embodiments, the person can be a normal person or a cancer patient. In some embodiments, the NK cells included in the composition can be autologous, i.e. obtained from the subject being treated.

In some embodiments, the NK cells disclosed herein and the cell therapeutic composition including the NK cells disclosed herein can be used for treating disease or condition other than cancer. It has been reported that NK cells plays an important role in the regulation of immune system, for example, by regulating of T-cells, thus the cell therapeutic composition having the NK cells can be administered to treat conditions associated with the immune system. For example, the cell therapeutic composition can be administered to treat neurodegenerative disorders (e g Alzheimer's disease and Parkinson's disease) or autoimmune diseases (e.g. rheumatoid arthritis, multiple sclerosis, psoriasis, spondyloarthropathies, SLE, Sjogren's syndrome, systemic sclerosis).

In some embodiments, a method of treating a subject can include collecting CD56+ cells from the subject; co-culturing the CD56+ cells with one or more feeder cells in the presence of IL-21; freezing the co-cultured CD56+ cells for at least a day; thawing the frozen CD56+ cells; expanding the thawed CD56+ cells; and administering the expanded CD56+ cells to the subject, wherein the cytotoxicity of the cells from the second expansion is at least 80% of a cytotoxicity of the co-cultured CD56+ before freezing (for example, at least 80, 85, 90, 95, or 97%).

In some embodiments, the method can further include storing the frozen CD56+ cells at a temperature lower than −100° C. In some embodiments, the frozen CD56+ cells may be stored at a temperature of −10° C. or lower, −20° C. or lower, −50° C. or lower, −70° C. or lower, −150° C. or lower, −192° C. or lower, or −200° C. or lower. In some embodiments, the frozen CD56+ cells can be stored for more than a day before thawing. In some embodiments, the frozen CD56+ cells can be stored for 2 days or more, 3 days or more, 7 days or more, 14 days or more, 30 days or more, 60 days or more, or 180 days or more, including any range between any two of the preceding values.

In some embodiments, the CD56+ cells can be co-cultured for between 13-16 days before freezing. For example, the CD56+ cells can be co-cultured for 14 or 15 days before freezing. In some embodiments, the co-culture or expansion (including re-expansion) can be for 9-25 days, e.g., 10-24, 11-23, 13-22, 14-21, 14-18, 14-16 days, etc. These time frames can be applied to any of the expansion and/or re-expansion periods provided herein (including for embodiments to other cells).

In some embodiments, expanding the thawed CD56+ cells comprises co-culturing the thawed CD56+ with one or more irradiated feeder cells in the presence of IL-21. The one or more feeder cells are one or more selected from a group consisting of irradiated Jurkat cells, irradiated Epstein-Barr virus transformed lymphocyte continuous line (EBV-LCL) cells, K562 cells and PBMCs. In some embodiments, the CD56+ cells can be co-cultured with a ratio of about 1:1-100 of CD56+ cells to feeder cells. For example, the CD56+ cells can be co-cultured with a ratio of about 1:2, 1:5, 1:10, 1:30 or 1:100 of CD56+ cells to feeder cells.

In some embodiments, IL-21 can be added at a concentration of 10-100 ng/mL during the first and/or second period. For example, IL-21 can be added at a concentration of 20-80 ng/mL, 30-70 ng/mL, or 40-60 ng/mL during the first and/or second period. In some embodiments, IL-21 can be added more than once during the first and/or second period.

In some embodiments, the IL-21 is human IL-21 for human NK cells.

Some embodiments provided herein include features and advantages of as follows:
(a) A method of producing natural killer cells.
(b) Since the natural killer cells can be produced in enough quantity for clinical use even after cryopreservation, it is possible to enhance an effect of prevention and treatment of cancer, particularly, allogenic therapy using the natural killer cells.
(c) The cytotoxicity of the resulting cells that have been expanded twice (at least the first time with IL-21, and optionally the second expansion as well) is surprisingly superior to cells expanded without IL-21 (once, and even greater twice). In some embodiments, the cells are in a 1-50 mL cryo-vial, or a 10˜100 mL cryo-bag.

### EXAMPLES

The following examples are provided to illustrate certain particular features and/or embodiments. These examples should not be construed to limit the disclosure to the particular features or embodiments described.

A comparison test for high-purity NK cell manufacturing method was performed using LCL+KL-1 types of feeder cell with and without IL-21 treatment. Any other feeder cells discussed in the present example is provide as a prophetic example.

The effectiveness of IL-21 in two manufacturing methods will be verified: 1) The original method, which involved culturing isolated CD56+ cells for 14-17 days with different types of feeder cells under treatment or non-treatment of IL-21 (but no subsequent re-expansion). 2) The re-stimulation method, which involved culturing cryopreserved expanded NK cells from the original method with different types of feeder cells under treatment or non-treatment of IL-21. The different types of feeder cells and manufacturing methods are shown in Table 1 and FIG. 1 and are understood to be representative of feeder cells in general for NK cell expansions.

TABLE 1
Feeder cell type and process for NK cell cultivation

| | | Innoculation | | |
|---|---|---|---|---|
| | | Ratio | | Re- |
| | | (NK cell: | Original | stimulation |
| Items | | Feeder cell) | method | method |
| Feeder | LCL + | 1:30:30 | Isolated CD56 | Cryopreserved |
| cell | KL-1 | | positive cells | NK cells at |
| type | cells | | from fresh or | Day 14 or 15 |
| | K562 | 1:10 | cryopreserved | |
| | cells | | PBMCs | |
| | PBMCs | 1:10 | | |

| | | |
|---|---|---|
| Culture | + IL-21 | Co-culture with irradiated feeder |
| Condition | | cells and IL-21 for 0 to 6 days |
| | −IL-21 | Co-culture with irradiated feeder |
| | | cells without IL-21 |

It is understood that the results provided by the present example are representative of feeder cells generally across a range of ratios, storage times, and additional ingredients (such as additional cytokines) as long as 11-21 is present.

### Example 1: Isolation of the Starting Material and Original Method

Peripheral blood mononuclear cells (PBMCs) were obtained from human blood. The isolated PBMCs were used for CD56+ cell selection. The PBMCs were isolated by density gradient centrifugation with 1.077 g/mL Ficoll, washed several times with PBS, and resuspended with AutoMACS Rinsing Solution (Miltenyi Biotec, Germany) with CD56 microbeads reagent. CD56+ cells were selected by using a magnetically activated cell sorting (MACS) system according to the manufacturer's instructions (Miltenyi Biotec, Germany) CD56+ selected cells were resuspended in the initial NK Cell Culture Medium with or without 50 ng/mL IL-21. Suspended cells were seeded into culture flasks after adding 100 Gy irradiated feeder cells, LCL+KL-1, K562 or PBMCs, and then cultured at 37° C. in 5% CO2 for 6 or 7 days. The specific condition for cell culture with each feeder type is as shown in the table below.

TABLE 2
Culture condition for NK cell expansion

| | Feeder cell ratio | Culture |
|---|---|---|
| Feeder | (CD56+ cells: | media and |
| cell type | Feeder cells) | supplement |
| LCL + | 1:30: 30 at | RPMI media, 10% FBS, 500 |
| KL-1 | D0 and D3 | IU/mL IL-2, 20 ug/mL |
| | | gentamicin |
| Autologous | 1:5 at D0 | CellGro SCGM medium, 1% |
| PBMCs¹⁾ | and D7 | autologous plasma or 10% |
| | | FBS, 10 ng/ml anti-CD3 |
| | | monoclonal antibody OKT3, |
| | | 500 IU/mL IL-2, 20 ug/mL |
| | | gentamicin |
| K562 | 1:10 | CellGro SCGM medium, 10 % |
| | | FBS, 500 IU/mL IL-2, 20 |
| | | ug/mL gentamicin |

### Reference: Immune Netw. 2018 Aug; 18(4):e31

At culture day 6 or 7, cells were collected from the culture flasks by centrifugation, and the cell number was assessed. The cells were resuspended with NK Cell Culture Medium, seeded into culture bag, and then cultured at 37° C. in 5% CO₂ up to 17 or18 days. Every 3 or 4 days, cells were sub-cultured with new media.

### Example 2: Cryopreservation of D14 Cells

At culture day 14 or 15, cells were harvested from the culture bag by centrifugation and the cell number assessed. The cells were resuspended in medium containing 90% FBS, 10% DMSO with or without 500 IU/mL IL-2, aliquoted into vials at a concentration of 5.0-10.0×10⁶ cells/mL, and then cells were cryopreserved at −196° C. liquid nitrogen tank for 1 week, 1, 3, 6, 12, 24 months and more.

### Example 3: Frozen Cell Thawing and Cell Culturing of Re-Stimulation Method

The cryo-preserved cells from the original method were thawed in a 37° C. water bath and resuspended in the initial NK Cell Culture Medium containing supplement for each feeder cell condition with or without 50 ng/mL IL-21. Suspended cells were seeded into culture flasks after adding 100 Gy irradiated feeder cells at, LCL+KL-1, K562 or autologous PBMCs, and then cultured at 37° C. in 5% CO2 for 6 or 7 days. This process was called to re-stimulation method. At culture day 6 or 7, cells were collected from the culture flasks by centrifugation, and the cell number assessed. The cells were resuspended with NK Cell Culture medium, seeded into culture bag, and then cultured at 37° C. in 5% CO₂ up to 17 or 18 days. Every 3 or 4 days, cells are sub-cultured with new media. The total cell culture period was taken 31˜33 days from the original method of DO to the final harvest through the re-stimulation process.

### Example 4: Population Doubling Level and Cell Growth

The NK cell culture following six different conditions were conducted using CD56+ cells based on the experiment design as illustrated in FIG. 1 and Table 3. It shows the result of experiment design type 1, as NK cells co-cultured with irradiated LCL and KL-1 as feeder cells. The particular conditions for the process, as shown in Table 3.

TABLE 3
Culture condition

| | | Re-Expansion After |
|---|---|---|
| | First step | Cry opreservation of |
| ID | expansion | Expanded NK Cells |
| IL21+. | (+) IL-21 | N/A |
| Type 1. IL21+/+ | (+) IL-21 | (+) IL-21 |
| Type 1. IL21+/− | (+) IL-21 | (−) IL-21 |
| IL21−. | (−) IL-21 | N/A |
| Type 1. IL21−/+ | (−) IL-21 | (+) IL-21 |
| Type 1. IL21−/− | (−) IL-21 | (−) IL-21 |

The cell expansion rate of NK cells was assessed by expansion fold relative to seeding cell number and by population doubling level (PDL) at each sub-culture day, calculated as 3.32 (log N−log No), where N is the number of cells at the end of each passage and No is the number of cells plated initially. The number of NK cells at every culture step was assessed by staining cells with trypan blue.

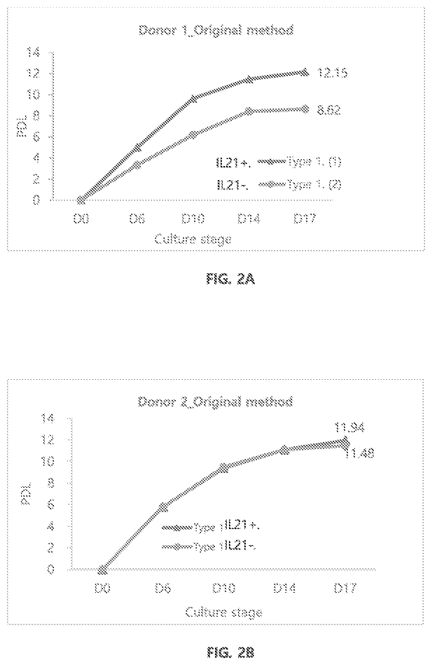

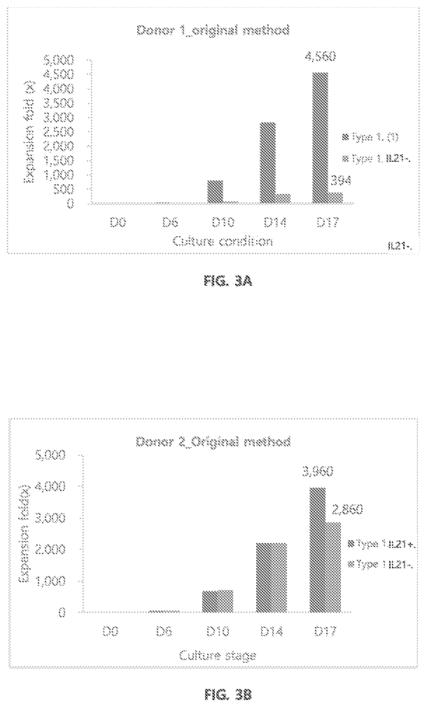

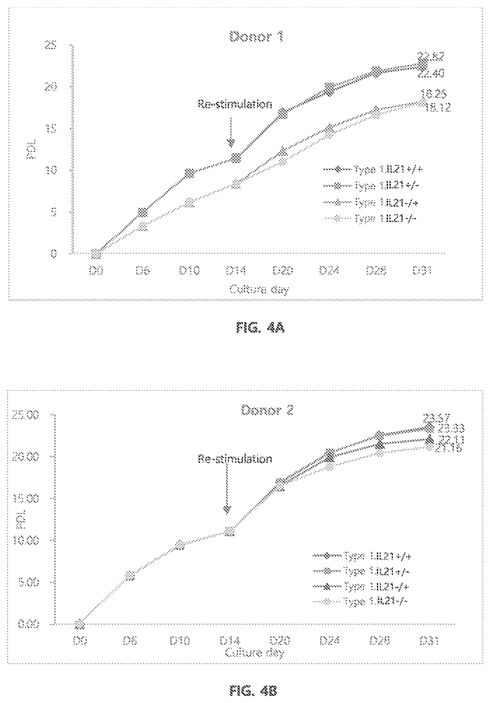

The production batches from two different donors were compared PDL and expansion fold. As shown the table 4, 5, and FIGS. 2A-3B, PDL and expansion fold of NK cells from one donor by the original method with IL-21 was shown highly growth rate than without IL-21. But, the batch from the other donor was shown a similar growth rate in both conditions.

TABLE 4
PDL of the original method

| Donor | ID | D0 | D6 | D10 | D14 | D17 |
|---|---|---|---|---|---|---|
| Donor 1 | IL21+. | 0.00 | 4.98 | 9.64 | 11.46 | 12.15 |
| | IL21−. | 0.00 | 3.32 | 6.17 | 8.40 | 8.62 |
| Donor 2 | IL21+. | 0.00 | 5.80 | 9.40 | 11.10 | 11.94 |
| | IL21−. | 0.00 | 5.80 | 9.49 | 11.10 | 11.48 |

TABLE 5
Expansion fold of the original method

| Donor | ID | D0 | D6 | D10 | D14 | D17 |
|---|---|---|---|---|---|---|
| Donor 1 | IL21+. | 1 | 32 | 800 | 2,833 | 4,560 |
| | IL21−. | 1 | 10 | 72 | 340 | 394 |
| Donor 2 | IL21+. | 1 | 56 | 680 | 2,200 | 3,960 |
| | IL21−. | 1 | 56 | 720 | 2,200 | 2,860 |

The cryopreserved cells at D14 from the original method were re-stimulated with feeder cells by treatment or non-treatment of IL-21, and cultured up to 17 or 18 days. The total cell culture period was taken 31˜33 days from the original method of D0 to the final harvest through the re-stimulation process (as shown in FIG. 1).

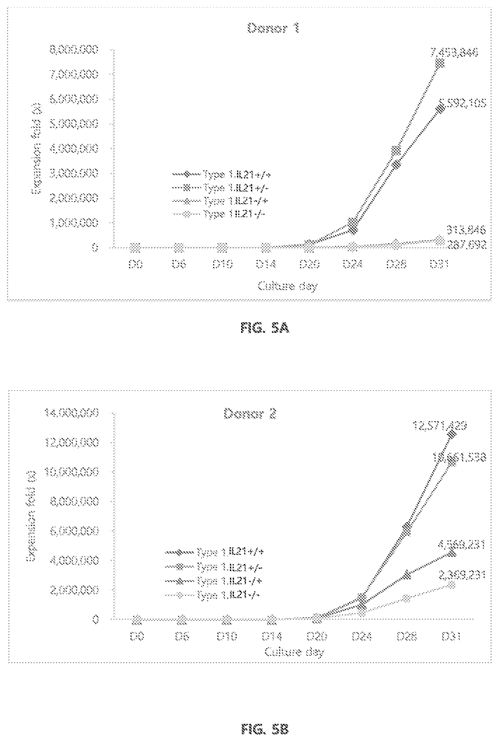

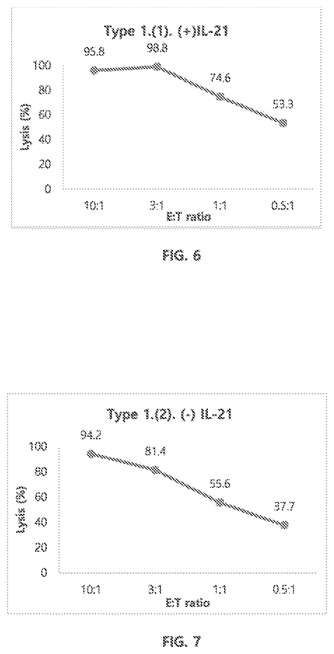

Several production batches from two donors were compared PDL and expansion fold. As shown in Tables 6, 7, and FIGS. 4A-5B, PDL and expansion fold of NK cells of the re-stimulation method with IL-21 provided higher growth rate than without IL-21 condition. Thus, IL-21, especially in the first round of expansion, is very useful in allowing for more effective subsequent expansion steps.

NK cells from Donor 2 demonstrated no difference in growth rate between with and without IL-21 in the original method, but the growth rate in IL-21 condition by the re-stimulation method was shown higher than without IL-21 condition at day 31.

TABLE 6
PDL of re-stimulation method

| Donor | ID | D0 | D6 | D10 | D14 | D20 | D24 | D28 | D31 |
|---|---|---|---|---|---|---|---|---|---|
| Donor | Type 1. | 0.00 | 4.98 | 9.64 | 11.46 | 16.94 | 19.42 | 21.67 | 22.40 |
| 1 | IL21+/+ | | | | | | | | |
| | Type 1. | 0.00 | 4.98 | 9.64 | 11.46 | 16.72 | 19.95 | 21.89 | 22.82 |
| | IL21+/− | | | | | | | | |
| | Type 1. | 0.00 | 3.32 | 6.17 | 8.40 | 12.35 | 15.15 | 17.25 | 18.25 |
| | IL21−/+ | | | | | | | | |
| | Type 1. | 0.00 | 3.32 | 6.17 | 8.40 | 11.02 | 14.25 | 16.63 | 18.12 |
| | IL21−/− | | | | | | | | |
| Donor | Type 1. | 0.00 | 5.80 | 9.40 | 11.10 | 16.89 | 20.47 | 22.57 | 23.57 |
| 2 | IL21+/+ | | | | | | | | |
| | Type 1. | 0.00 | 5.80 | 9.40 | 11.10 | 16.94 | 20.53 | 22.48 | 23.33 |
| | IL21+/− | | | | | | | | |
| | Type 1. | 0.00 | 5.80 | 9.49 | 11.10 | 16.50 | 19.94 | 21.53 | 22.11 |
| | IL21−/+ | | | | | | | | |
| | Type 1. | 0.00 | 5.80 | 9.49 | 11.10 | 16.50 | 18.79 | 20.44 | 21.16 |
| | IL21−/− | | | | | | | | |

TABLE 7
Expansion fold of re-stimulation method

| Donor | ID | D0 | D6 | D10 | D14 | D20 | D24 | D28 | D31 |
|---|---|---|---|---|---|---|---|---|---|
| Donor | Type 1. | 1 | 32 | 800 | 2,833 | 126,754 | 708,333 | 3,355,263 | 5,592,105 |
| 1 | IL21+/+ | | | | | | | | |
| | Type 1. | 1 | 32 | 800 | 2,833 | 108,974 | 1,024,359 | 3,923,077 | 7,453,846 |
| | IL21+/− | | | | | | | | |
| | Type 1. | 1 | 10 | 72 | 340 | 5,231 | 36,615 | 156,923 | 313,846 |
| | IL21−/+ | | | | | | | | |
| | Type 1. | 1 | 10 | 72 | 340 | 2,092 | 19,615 | 102,000 | 287,692 |
| | IL21−/− | | | | | | | | |
| Donor | Type 1. | 1 | 56 | 680 | 2,200 | 122,222 | 1,466,667 | 6,285,714 | 12,571,429 |
| 2 | IL21+/+ | | | | | | | | |
| | Type 1. | 1 | 56 | 680 | 2,200 | 126,923 | 1,523,077 | 5,923,077 | 10,661,538 |
| | IL21+/− | | | | | | | | |
| | Type 1. | 1 | 56 | 720 | 2,200 | 93,077 | 1,015,385 | 3,046,154 | 4,569,231 |
| | IL21−/+ | | | | | | | | |
| | Type 1. | 1 | 56 | 720 | 2,200 | 93,077 | 456,923 | 1,438,462 | 2,369,231 |
| | IL21−/− | | | | | | | | |

### Example 5: Purity of NK Cells

NK cells are known to express CD56 and lack CD3. To investigate the purity of NK cells before expansion and after expansion by the original and re-stimulation method, flow cytometric analysis were applied. NK cells were stained with fluorochrome-labeled antibodies of anti-CD56-FITC and anti-CD3-PE, and then analyzed by using flow cytometry.

As the NK cell culture progressed by the original and re-stimulation method in both conditions (with and without IL-21 treatment), the proportion of NK cells (CD56+CD3−) rapidly increased in expanded NK cells from 2 different donors with over 99% at day 31 (Table 7). Other cell type of cell surface marker, such as CD3+, CD20+, and CD14+were shown the very low population at the final culture stage (Table 8).

TABLE 8
NK cell marker expression (CD3−CD56+) pattern on culture-
expanded NK cells

| | | Before Frozen | | | | After Thawing | | |
|---|---|---|---|---|---|---|---|---|
| Donor | ID | D0 | D10 | D14 | D17 | D24 | D28 | D31 |
| Donor | Type 1. | 75.23 | 98.31 | 99.24 | 99.27 | 99.35 | 99.65 | 99.60 |
| 1 | IL21+/+ | | | | | | | |
| | Type 1. | — | — | — | — | 98.67 | 99.24 | 99.70 |
| | IL21+/− | | | | | | | |
| | Type 1. | 75.23 | 94.30 | 96.29 | 95.63 | 90.93 | 96.38 | 98.09 |
| | IL21−/+ | | | | | | | |
| | Type 1. | — | — | — | — | 94.42 | 92.59 | 97.28 |
| | IL21−/− | | | | | | | |
| Donor | Type 1. | 82.83 | 98.60 | 98.20 | 98.83 | 98.68 | 99.36 | 99.86 |
| 2 | IL21+/+ | | | | | | | |
| | Type 1. | — | — | — | — | 98.16 | 99.69 | 99.84 |
| | IL21+/− | | | | | | | |
| | Type 1. | 82.83 | 99.03 | 98.64 | 99.39 | 96.84 | 99.47 | 99.81 |
| | IL21−/+ | | | | | | | |
| | Type 1. | — | — | — | — | 97.43 | 99.21 | 99.63 |
| | IL21−/− | | | | | | | |

TABLE 9
Other cell marker expression (CD3, CD20, CD14) pattern on
culture-expanded NK cells

| | | Surface | Before Frozen | | | | After Thawing | | |
|---|---|---|---|---|---|---|---|---|---|
| Donor | ID | Marker | D0 | D10 | D14 | D17 | D24 | D28 | D31 |
| Donor | Type 1. | CD3 | 24.1 | 0.38 | 0.2 | 0.48 | 0.28 | 0.06 | 0.22 |
| 1 | IL21+/+ | CD20 | 0.43 | 0.04 | 0.68 | 0.45 | 0.57 | 0.12 | 0.05 |
| | | CD14 | 0.28 | 0.30 | 1.72 | 0.23 | 0.12 | 0.12 | 0.19 |
| | Type 1. | CD3 | — | — | — | — | 0.13 | 0.11 | 0.08 |
| | IL21+/− | CD20 | — | — | — | — | 0.16 | 0.14 | 0.05 |
| | | CD14 | — | — | — | — | 0.03 | 0.27 | 0.23 |
| | Type 1. | CD3 | 24.1 | 2.03 | 2.21 | 3.38 | 0.85 | 0.26 | 0.61 |
| | IL21−/+ | CD20 | 0.43 | 0.54 | 1.11 | 0.38 | 0.45 | 0.59 | 0.14 |
| | | CD14 | 0.28 | 1.85 | 1.75 | 0.61 | 0.28 | 0.17 | 0.44 |
| | Type 1. | CD3 | — | — | — | — | 0.95 | 1.13 | 1.40 |
| | IL21−/− | CD20 | — | — | — | — | 0.88 | 0.51 | 0.21 |
| | | CD14 | — | — | — | — | 0.66 | 0.13 | 0.38 |
| Donor | Type 1. | CD3 | 16.96 | 1.13 | 1.40 | 1.08 | 0.86 | 0.41 | 0.10 |
| 2 | IL21+/+ | CD20 | 1.35 | 0.34 | 0.20 | 0.02 | 0.49 | 0.45 | 0.04 |
| | | CD14 | 0.47 | 0.25 | 0.20 | 0.04 | 0.53 | 0.27 | 0.05 |
| | Type 1. | CD3 | — | — | — | — | 1.08 | 0.14 | 0.10 |
| | IL21+/− | CD20 | — | — | — | — | 0.85 | 0.12 | 0.03 |
| | | CD14 | — | — | — | — | 0.60 | 0.00 | 0.08 |
| | Type 1. | CD3 | 16.96 | 0.59 | 1.08 | 0.55 | 0.87 | 0.11 | 0.07 |
| | IL21−/+ | CD20 | 1.35 | 0.15 | 0.06 | 0.12 | 0.80 | 0.13 | 0.06 |
| | | CD14 | 0.47 | 0.22 | 0.10 | 0.13 | 0.57 | 0.12 | 0.05 |
| | Type 1. | CD3 | — | — | — | — | 1.30 | 0.62 | 0.35 |
| | IL21−/− | CD20 | — | — | — | — | 1.17 | 0.54 | 0.13 |
| | | CD14 | — | — | — | — | 1.05 | 0.64 | 0.31 |

### Example 6 Cytotoxic Function of NK Cells

The cytotoxicity of NK cells against tumor target cell lines was assessed by fluorometric cytotoxicity assay. NK cells was co-cultured with K-562 cell that is stained with Calcein AM at the E:T ratios of 10:1, 3:1, 1:1, and 0.5:1 for 4 hours under light protection. RPMI1640 containing 10% FBS or 2% triton X100 was added to the target cells to provide spontaneous and maximum release. For the Calcein release assay, the supernatant after incubation of NK cells with target cells is recovered, and its fluorescence is assessed using a SpectraMax M2 microplate reader (Molecular devices, San Jose, CA). The percent specific lysis is calculated using the formula [(Test release−Spontaneous release)/(Maximum release−Spontaneous release)]×100.

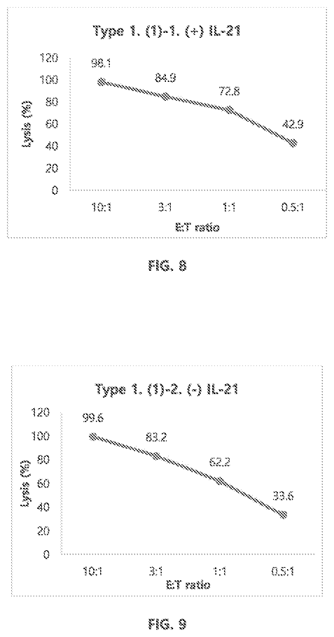

The cytotoxicity of cultured NK cells by the original and re-stimulation method was tested using the standard K-562 cell line, which is a NK sensitive target. The expanded cells from the original method with and without IL-21 condition exerted strong cytotoxic activity against K-562 even at low E:T ratios (1:1 and 0.5:1) (FIGS. 6-7). But, the cytotoxic activity of NK cells by the re-stimulation method was shown different level on different conditions with and without IL-21 treatment. The NK cells by IL-21 treatment for both original and re-stimulation methods were exerted more strong cytotoxic activity than other conditions (FIGS. 8-11).

But the NK cells by non-treatment IL-21 condition for both original and re-stimulation condition was decreased cytotoxic activity at E:T ratios of 0.05:1 to 3:1. Also, there was shown a lower level of cytotoxicity at E:T ratio of 10:1 than the IL-21 treatment condition.

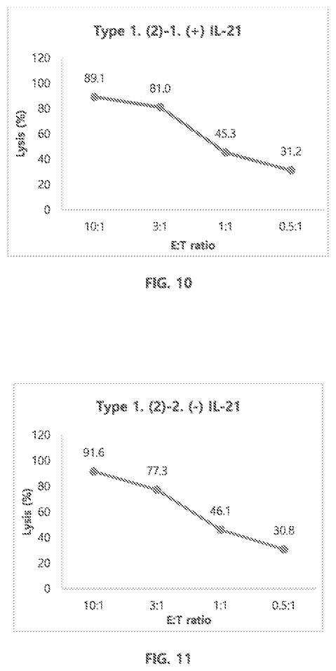

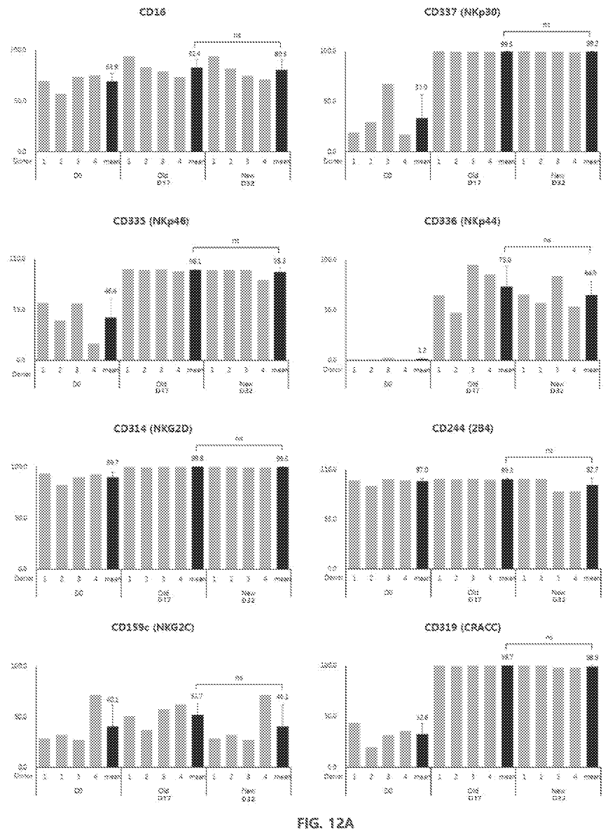

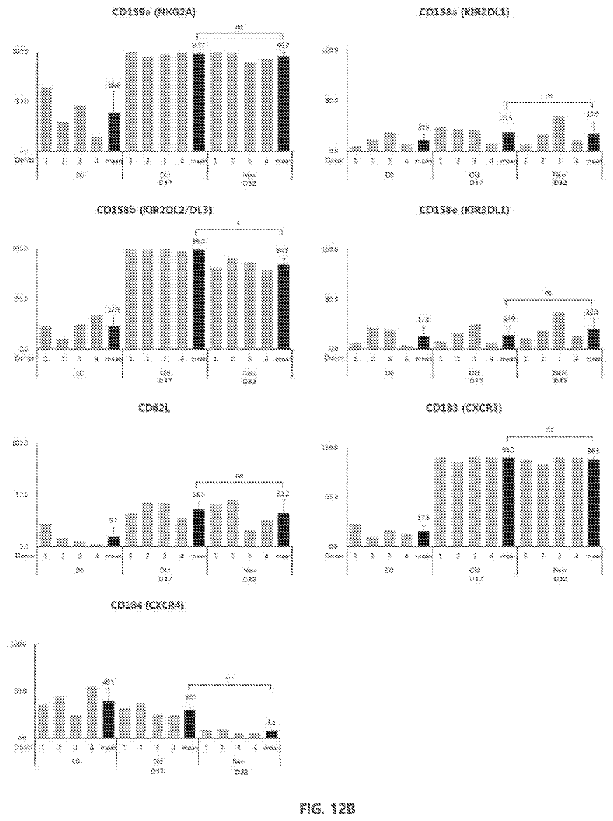

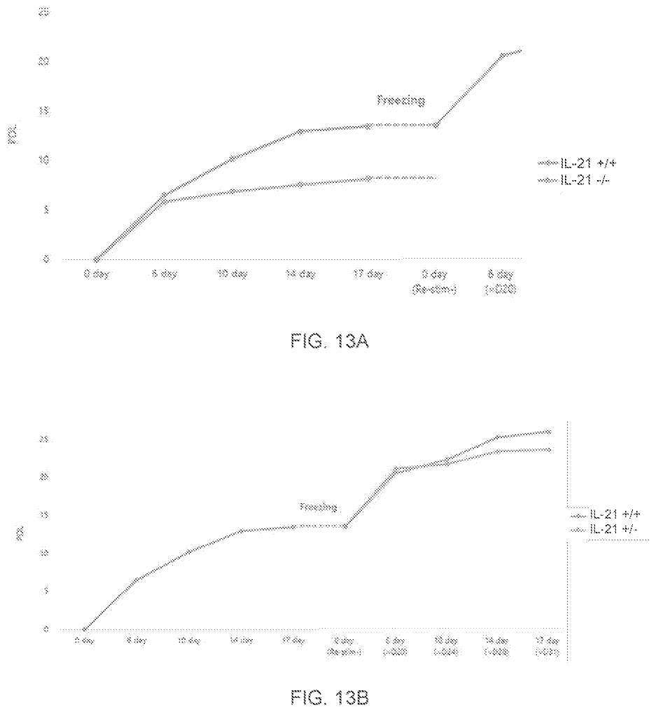

Expanded NK cells with IL-21 treatment showed highly potent cytotoxicity against K-562 cell line, with a similar cytotoxicity in both NK cells manufactured by the original and re-stimulation method (FIGS. 6-11). FIG. 6 displays the cytotoxic activity of NK cells against K562 cells expanded with IL-21 (IL-21+). FIG. 7 displays the cytotoxic activity of NK cells against K562 cells expanded without IL-21 (IL-21−). FIG. 8 displays the cytotoxic activity of NK cells against K562 cells expanded with IL-21 and re-stimulated with IL-21 (IL-21+/+). FIG. 9 displays the cytotoxic activity of NK cells against K562 cells expanded with IL-21 and re-stimulated without IL-21(IL-21+/−). FIG. 10 displays the cytotoxic activity of NK cells against K562 cells expanded without IL-21 and re-stimulated with IL-21(IL-21−/+). FIG. 11 displays the cytotoxic activity of NK cells against K562 cells expanded without IL-21 and re-stimulated without IL-21(IL-21−/−). FIG. 12A displays the phenotypic comparisons of NK cells' Activating receptors. FIG. 12B displays the phenotypic comparisons of NK cells' inhibitory and chemokine receptors.

### Surface Marker Expression

NK cell function is finely regulated by the balance between activating and inhibitory receptors expressed on their surface. To phenotypically characterize the expression levels of activating [CD16, NKp30, NKp46, NKp44, NKG2D, 2B4 (CD244), NKG2C, CRACC] or inhibitory NK receptors [NKG2A, KIR: CD158a (KIR2DL1), CD158b (KIR2DL2/L3), CD158e (KIR3DL1)], chemokine receptors (CXCR3, CXCR4), or adhesion molecule (CD62L) was analyzed on the gated CD56+ NK cells before expansion (Day 0; D0) and after expansion by the original (Old) process and re-stimulation process with IL-21 treatment for 17-18 days. Surface receptor expression level was calculated as percentage of receptor-positive subsets of NK cells in samples. NK cells at each stage of original and re-stimulation process are stained with fluorochrome-labeled antibodies of each marker, and then analyzed by using flow cytometry.

Surface receptor expression level has analyzed the cells from the starting and final stage (D0, D17, and D32) that has cultured by original and re-stimulation process with IL-21 treatment in type 1 experiment for 4 different donors. Surface receptor expression level was calculated as percentage of receptor-positive subsets of NK cells in samples.

Among activating receptors, the expression level of CD16, NKp30, NKp46, NKp44, NKG2D, and CRACC was increased during the expansion of NK cells manufactured by the original process (Old) and was similar with expanded NK cells by the re-stimulation process (New), whereas that of NKG2C and 2B4 was not changed after culture expansion by both methods (FIG. 6). Expression of the inhibitory receptors, CD158a and CD158e remained largely unchanged upon culture by both process (Old and New) while proportions of NKG2A and CD158b were significantly increased (FIGS. 12A-B). All inhibitory receptor expression level analyzed was similar on NK cells manufactured by both methods. Expression of chemokine receptors such as CXCR3 and CXCR4 was also evaluated.

The frequency of CXCR3+ NK cells was significantly increased during NK cell expansion by both methods (Old and New) with similar expression levels whereas the frequency of CXCR4+ NK cells was decreased after culture expansion by both methods, with a slightly more decrease on NK cells manufactured by the new method (FIGS. 12A-B). The expression level of CD62L was slightly increased on the expanded NK cells by both methods.

As demonstrated by these results, the nature of IL21+ and IL21+/+ expanded cells are similar, despite the fact that one set has gone through a re-expansion procedure.

### Example 7: IL-21 Concentration

For various IL-21 concentration, isolated CD56+ cells were resuspended in the initial NK Cell Culture Medium containing 10% FBS, 500 IU/mL IL-2, 20 μg/mL gentamicin in RPMI medium with 10, 30, 50, and 100 ng/mL IL-21 of type 1 experiment design (IL21+ only, no re-stimulation performed).

### Example 8: IL-21 Concentration

In type 1 experiment was performed with different ratio of feeder cells at 1:10:10 (CD56+ cells:LCL:KL-1), and 1:20:20, 1:30:30 (IL21+only, no re-stimulation performed).

### Example 9

This non-limiting example shows IL-21 enhancing expansion of CD56+ and CD3−/CD56+ NK cells with freeze-thawing.

### (1) Preparation of CD56+ Natural Killer Cells (NK Cells)-1

First, blood PBMC were isolated using a Ficoll density gradient (Ficoll-Hypaque density gradient method). The PBMC were further treated according to 1-1 or 1-2 below.

1-1. CD56+ cell isolation

The PBMC were suspended by addition of MACS buffer (1×PBS+0.5% HSA) and CD56 microbeads (Miltenyi Biotec) were added to obtain 1-20 μL per 1.0×10⁷ PBMC and incubated for 5 - 30 minutes at 2-8° C. After incubation, MACS buffer was added and mixed, and the mixture was centrifuged (600 xg) to precipitate the cells. After centrifugation, the supernatant was removed and MACS buffer was added to resuspend the cells, and the cells were added to a MACS separator coupled to a column. MACS buffer was passed through the column to remove non-specific binding. The column was separated from the MACS separator and transferred to a 15 mL conical tube, and MACS buffer was added to isolate CD56+ cells attached to the column.

### 1-2. CD3−/CD56+ Cell Isolation

CD3−/CD56+ cells were isolated as follows. The PBMC were suspended by addition of MACS buffer (1×PBS+0.5% HSA) and CD3 microbeads (Miltenyi Biotec) were added to obtain 1-20 μL per 1.0×10⁷ PBMC and incubated for 5-30 minutes at 2-8° C. After incubation, MACS buffer was added and mixed, and the mixture was centrifuged (600 xg) to precipitate the cells. After centrifugation, the supernatant was removed and MACS buffer was added to resuspend the cells, and the cells were added to a MACS separator coupled to a column. MACS buffer was passed through the column to recover CD3− cells.

The MACS buffer (1×PBS+0.5% HSA) was added to the recovered CD3− cells to resuspend the CD3− cells and CD56 microbeads (Miltenyi Biotec) were added to obtain 1-20 pL per 1.0×10⁷ CD3− cells and incubated at 2 to 8° C. for 5 to 30 minutes. After incubation, MACS buffer was added and mixed, and the mixture was centrifuged (600 xg) to precipitate the cells. After centrifugation, the supernatant was removed and MACS buffer was added to resuspend the cells, and the cells were added to a MACS separator coupled to a column. MACS buffer was passed through the column to remove non-specific binding. The column was separated from the MACS separator and transferred to a 15 mL conical tube, and MACS buffer was added to isolate CD3−/CD56+ cells attached to the column.

### 1-3. Primary Culture

The separated CD56 + cells or CD3−/CD56+ cells from 1-1 and 1-2 were each co-cultured in an incubator with feeder cells (Jurkat cells, and EBV-LCL cells) previously prepared by 100 Gy irradiation with and in the presence of IL-2 and IL-21 at 500 IU/mL and 50 ng/mL concentration, respectively, in RPMI-1640 medium with 10% FBS at 37° C., 5% CO².

On day 6, cells were inoculated at 1.0×10⁵-2.0×10⁶/mL in a 350 mL standard bag and cultivated for four additional days, and on day 10 the cells were inoculated at 1.0×10⁵-2.0×10⁶ cells/mL, in a 1L bag and cultured for another 4 days. At this time, the ratio (CD56+ cells or CD3−/CD56+ cells):(Jurkat cell):(EBV-LCL cell) is 1:30:30 during incubation.

### 1-4. Secondary Culture After Freezing and Thawing

On the fourteenth day of culture 1 (1-3), the cultured cells were suspended in a solution containing 90% FBS and 10% DMSO, stored frozen at −192° C. or lower, and thawed in a 37° C. constant temperature water bath according to the culture schedule.

Then, RPMI-1640 containing 10% of FBS to which IL-2 and IL-21 were added at a concentration of 500 IU/mL and 50 ng/mL, respectively, along with 100 Gy irradiated feeder cells (Jurkat cells and EBV-LCL cells). After putting in the medium, it was co-cultured in a 37° C., 5% CO 2 incubator.

On day 6 after thawing and culturing, the cells were inoculated into a 350 mL bag (at 1.0×10⁵-2.0×10⁶ cells/mL and incubated for an additional four days, and on day 10 the cells were inoculated into a 1L standard bag at 1.0×10⁵-2.0×10⁶ cells/mL and further cultured for 4 days.

In order to sustain the growth of cells in culture until the 14th day after thawing, cells were co-cultured with 100 Gy irradiated feeder cells (Jurkat cells and EBV-LCL cells) in the presence of IL-2 and IL-21 at a concentration of 500 IU/mL and 50 ng/mL, respectively. Cells were cultured in RPMI-1640 medium containing 10% of added FBS, in an incubator at 37° C., 5% CO².

On the 20th day after thawing, the cells were inoculated into a 1L bag at 1.0×10⁵-2.0×10⁶ cells/mL, followed by additional culture for 4 days, and on the 24th day of culture after thawing, the cells were inoculated into a 1L bag at 1.0×10⁵-2.0×10⁶ cells/mL and further cultured for 4 days.

Finally, on the 28th day of culture after thawing, the cells were inoculated into a 1L bag at 1.0×10⁵-2.0×10⁶ cells/mL, followed by additional culture for 3-6 days. At this time, (CD56+ cells or CD3−/CD56+ cells):(Jurkat cells):(EBV-LCL cells) were cultured at a ratio of 1:30:30.

### (2) Preparation of CD56+ Natural Killer Cells-2

Natural killer cells were prepared in the same manner as in (1), except for the step of adding cytokines in 1-4.

(3) Comparative Example. Preparation of Natural Killer Cells Excluding Cytokine Treatment Steps

Natural killer cells were prepared in the same manner as in (1), except for the step of adding cytokine (IL-21) in 1-3 and 1-4 .

### (4) Confirmation of NK Cell Proliferation Ability

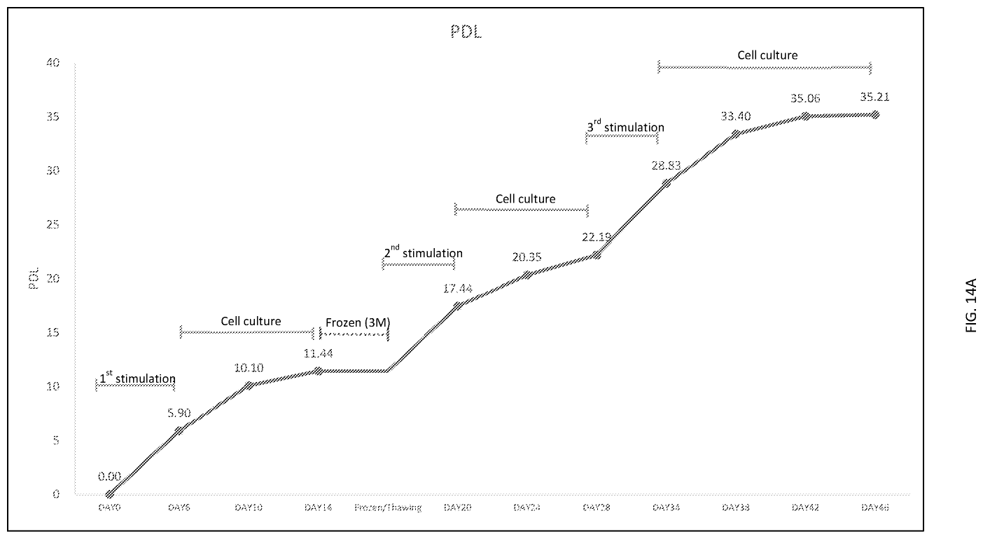

The proliferative ability of NK cells cultured by the methods of (1)-(3) were measured. As can be seen in FIG. 13A, when the cytokine was not treated during the primary culture (IL-21 −/−); see (3) above), it was found that a sufficient number of NK cells for clinical application was not produced after the freezing and thawing process (FIG. 13A). On the other hand, when the cells were treated with cytokine (IL-21 +/+; see (1) above), NK cells were produced in sufficient numbers for clinical application even after the freezing and thawing process, and these results were not only when the cytokine was treated after the freezing and thawing process. In the case where cells were not treated with cytokine after freezing and thawing (IL-21 +/−; see (2) above), the NK cells expanded as well as those treated with cytokine after freezing and thawing (FIG. 13B).

### Example 10

Further results of some embodiments of the various expansion and freezing and re-freezing processes are shown in FIGS. 14A and 14B. FIG. 14A shows the resulting PDL (Population doubling level) and depicts some embodiments of the different periods during expansion. FIG. 14B depicts the resulting expansion fold for the example.

This Example analyzes whether NK Cells could be stimulated or expanded multiple times after being frozen at least once, and whether the proliferation of NK cells could be stopped and restarted, rather than simply being maintained. NK cells were first cultured 14 days. The NK cells were treated with IL-21 (50 ng/mL) and feeder cells twice at 3 days interval, during the first six-day period (Day 0-6).

After this first 14-day expansion the cells were Frozen for 90 days, then thawed to be cultured for another 14 days. The NK cells were treated again with IL-21 (50 ng/mL) and feeder cells twice at 3 days interval, during the first six-day period (Day 0-6) during this second expansion.

The cells were finally re-cultured for 18 days by re-stimulated them a third time with IL-21 (50 ng/mL) and feeder cells twice at 3 days interval, during the first six-day period (Day 0-6) during this second expansion.

The expansion of NK cells were monitored for the 46 days of culture. As shown in FIGS. 14A and 14B, when treated with feeder cells and IL-21 twice or more, even after the cells were frozen, the NK cells exhibited significant expansion after each stimulation.

### Terminology

The foregoing description of the exemplary embodiments has been presented only for the purposes of illustration and description and is not intended to be exhaustive or to limit the invention to the precise forms disclosed. Many modifications and variations are possible in light of the above teaching. It is contemplated that various combinations or sub combinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. The ranges disclosed herein also encompass any and all overlap, sub-ranges, and combinations thereof.

The embodiments were chosen and described in order to explain the principles of the invention and their practical application so as to activate others skilled in the art to utilize the invention and various embodiments and with various modifications as are suited to the particular use contemplated. Alternative embodiments will become apparent to those skilled in the art to which the invention is defined by the appended claims rather than the foregoing description and the exemplary embodiments described therein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

The ranges disclosed herein also encompass any and all overlap, sub-ranges, and combinations thereof. Language such as “up to,” “at least,” “greater than,” “less than,” “between,” and the like includes the number recited.

Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

The term “generally” as used herein represents a value, amount, or characteristic that predominantly includes or tends toward a particular value, amount, or characteristic. As an example, in certain embodiments, the term “generally uniform” refers to a value, amount, or characteristic that departs from exactly uniform by less than 20%, less than 15%, less than 10%, less than 5%, less than 1%, less than 0.1%, and less than 0.01%.

The ranges disclosed herein also encompass any and all overlap, sub-ranges, and combinations thereof. Language such as “up to,” “at least,” “greater than,” “less than,” “between” and the like includes the number recited. Numbers preceded by a term such as “about” or “approximately” include the recited numbers. For example, “about 5.0 cm” includes “5.0 cm.”

Some embodiments have been described in connection with schematic drawings. However, it should be understood that the schematic drawings are not drawn to scale. Distances are merely illustrative and do not necessarily bear an exact relationship to actual dimensions and layout of the devices illustrated.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

What is claimed is:

1. A method of expanding natural killer cells in culture, comprising:
isolating CD56+ cells from a blood sample;
co-culturing the isolated CD56+ cells in the presence of IL-21 for a first period;
freezing the co-cultured CD56+ cells after the first period;
thawing the frozen CD56+ cells; and
co-culturing the thawed CD56+ cells in the presence of IL-21 for a second period.

2. The method of claim 1, further comprising storing the frozen CD56+ cells at a temperature lower than −100° C.

3. The method of claim 1 or 2, further comprising storing the frozen CD56+ cells for more than a day before thawing.

4. The method of any of the preceding claims, wherein the isolated CD56+ cells is co-cultured for between 13-16 days before freezing.

5. The method of any of the preceding claims, wherein the isolated CD56+ cells are co-cultured with one or more irradiated feeder cells in the presence of IL-21.

6. The method of any of the preceding claims, wherein the thawed CD56+ cells are co-cultured with one or more irradiated feeder cells in the presence of IL-21.

7. The method of claim 5 or 6, wherein one or more feeder cells are one or more selected from a group consisting of irradiated Jurkat cells, irradiated Epstein-Barr virus transformed lymphocyte continuous line (EBV-LCL) cells, K562 cells and PBMCs.

8. The method of any one of claims 5-7, wherein the CD56+ cells are co-cultured with a ratio of about 1:1-100 of CD56+ cells to the feeder cells.

9. The method of claim 8, wherein the CD56+ cells are co-cultured with a ratio of about 1:2 of CD56+ cells to feeder cells.

10. The method of claim 8, wherein the CD56+ cells are co-cultured with a ratio of about 1:5 to 1:30 of CD56+ cells to feeder cells.

11. The method of claim 8, wherein the CD56+ cells are co-cultured with a ratio of about 1:10 of CD56+ cells to feeder cells.

12. The method of claim 8, wherein the CD56+ cells are co-cultured with a ratio of about 1:30 of CD56+ cells to feeder cells.

13. The method of claim 8, wherein the CD56+ cells are co-cultured with a ratio of about 1:1-100 of CD56+ cells to feeder cells.

14. The method of any of the preceding claims, wherein IL-21 is added at a concentration of 10-100 ng/mL during the first and/or second period.

15. The method of any one of claims 1-13, wherein IL-21 is added at a concentration of 20-80 ng/mL during the first and/or second period.

16. The method of any one of claims 1-13, wherein IL-21 is added at a concentration of 30-70 ng/mL during the first and/or second period.

17. The method of any of the preceding claims, wherein IL-21 is added more than once during the first and/or second period.

18. A method of expanding natural killer cells in culture, comprising:
isolating CD56+ from a blood sample;
co-culturing the CD56+ cells with one or more feeder cells in the presence of IL-21;
freezing the CD56+ cells;
thawing the frozen CD56+ cells; and
expanding the thawed CD56+ cells.

19. The method of claim 18, wherein freezing the CD56+ cells at a temperature lower than −100° C.

20. The method of claim 18 or 19, further comprising storing the frozen CD56+ cells for a period more than a day and less than 10 years

21. The method of any one of claims 18-20, wherein the CD56+ cells is co-cultured for between 13-16 days before freezing.

22. The method of any one of claims 18-21, wherein one or more feeder cells are one or more selected from a group consisting of irradiated Jurkat cells, irradiated Epstein-Barr virus transformed lymphocyte continuous line (EBV-LCL) cells, K562 cells and PBMCs.

23. The method of any one of claims 18-22, wherein the CD56+ cells are co-cultured with a ratio of about 1:1-100 of CD56+ cells to feeder cells.

24. The method of any of claims 18-23, wherein IL-21 is added at a concentration of 10-100 ng/mL.

25. The method of any of claims 18-24, wherein IL-21 is added more than once.

26. A method of increasing cytotoxicity of natural killer cells, comprising:
providing said natural killer cells;
freezing said natural killer cells;
thawing the frozen natural killer cells; and
co-culturing the thawed natural killer cells with one or more feeder cells in the presence of IL-21.

27. The method of claim 26, further comprising storing the frozen natural killer cells at a temperature lower than −100° C.

28. The method of claim 26 or 27, further comprising storing the frozen natural killer cells for more than a day before thawing.

29. The method of any one of claims 26-28, wherein one or more feeder cells are one or more selected from a group consisting of irradiated Jurkat cells, irradiated Epstein-Barr virus transformed lymphocyte continuous line (EBV-LCL) cells, K562 cells and PBMCs.

30. The method of any one of claims 26-29, wherein the thawed natural killer cells are co-cultured with a ratio of about 1:1-100 of CD56+ cells to feeder cells.

31. The method of any one of claims 26-30, wherein IL-21 is added at a concentration of 10-100 ng/mL.

32. The method of any one of claims 26-31, wherein IL-21 is added more than once.

33. A method of treating a subject comprising:
collecting CD56+ cells from the subject;
co-culturing the CD56+ cells with one or more feeder cells in the presence of IL-21;
freezing the co-cultured CD56+ cells for at least a day;
thawing the frozen CD56+ cells;
expanding the thawed CD56+ cells; and
administering the expanded CD56+ cells to the subject, wherein the cytotoxicity of the cells from the second expansion is at least 80% of a cytotoxicity of the co-cultured CD56+ cells before freezing.

34. The method of claim 33, further comprising storing the frozen CD56+ cells at a temperature lower than −100° C.

35. The method of claim 33 or 34, further comprising storing the frozen CD56+ cells for more than a day before thawing.

36. The method of any one of claims 33-35, wherein the isolated CD56+ cells is co-cultured for between 13-16 days before freezing.

37. The method of any one of claims 33-36, wherein expanding the thawed CD56+ cells comprises co-culturing the thawed CD56+ with one or more irradiated feeder cells in the presence of IL-21.

38. The method of any one of claims 33-37, wherein one or more feeder cells are one or more selected from a group consisting of irradiated Jurkat cells, irradiated Epstein-Barr virus transformed lymphocyte continuous line (EBV-LCL) cells, K562 cells and PBMCs.

39. The method of any one of claims 33-38, wherein the CD56+ cells are co-cultured with a ratio of about 1:1-100 of CD56+ cells to feeder cells.

40. The method of any one of claims 33-39, wherein IL-21 is added at a concentration of 10-100 ng/mL during the first and/or second period.

41. The method of any one of claims 33-40, wherein IL-21 is added more than once during the first and/or second period.

42. A composition comprising:
an effective amount of CD56+ cells derived from peripheral blood mononuclear cells (PBMCs) from the patient, wherein the CD56+ cells are prepared by:
isolating peripheral blood mononuclear cells (PBMCs) from a blood sample;
isolating CD56+ cells from the PBMCs;
co-culturing the CD56+ cells with one or more feeder cells in the presence of one or more cytokines;
freezing the CD56+ cells;
thawing the frozen CD56+ cells; and
co-culturing the thawed CD56+ cells with one or more feeder cells in the presence of one or more cytokines.

43. A cell composition comprising:
an effective amount of CD56+ cells derived from peripheral blood mononuclear cells (PBMCs) from the patient;
IL-2; and
IL-21.

44. A composition comprising:
a first population of CD56+ cells derived from peripheral blood mononuclear cells (PBMCs);
ice; and
IL-2
IL-21,
wherein, when thawed, the CD56+ cell has a cytotoxicity of at least 80% of a second population of CD56+ cells, wherein the second population of CD56+ cells have not been frozen.

45. A method of expanding natural killer cells in culture, comprising:
providing PBMCs;
co-culturing the PBMCs in the presence of IL-21 for a first period;
freezing the co-cultured PBMCs after the first period;
thawing the frozen PBMCs; and
co-culturing the thawed PBMCs in the presence of IL-21 for a second period.

46. The method of claim 45, wherein a PBMC ratio to feeder cells is 1:0.5:0.5.

47. The method of any of claims 2-17, but as dependent from claim 45, rather than claim 1.

48. The method of claim 33, further comprising freezing in a ready-to-inject solution.

49. The method of claim 33 or 34, wherein the cytotoxicity is comparing a non-frozen expansion (with and without IL-21) versus one that was frozen but co cultured with IL-21+ on the first step, wherein an average cytotoxicity of the frozen expansion is 97.7% of the non-frozen.

50. The method of claim 33 or 34, wherein the cytotoxicity is comparing a non-frozen expansion (with and without IL-21) versus one that was frozen but not co cultured with IL-21+on the first step, wherein an average cytotoxicity of the frozen expansion is 81.4% of the non-frozen expansion.

51. The method of claim 33 or 34, wherein, IL-21 is added both before and after freezing, and wherein an average cytotoxicity is 114% of a non-frozen expansion.

52. A composition comprising:
IL-2;
5-10% DMSO;
90-95% FBS; and
NK cells that are optionally CD56+ cells.

53. The composition of claim 52, wherein the composition is frozen solid.

54. The composition of claim 52, wherein the NK cells are at least 90% of a cell population of the composition.

55. A composition comprising:
IL-2;
5-10% DMSO;
80-95% Hartman solution;
1-10% human serum albumin; and
NK Cells.

56. The composition of any one of claims 52-55, further comprising CryoStor solution.
